SEQUENCE LISTING (34 sequences)

SEQ ID NO: 1 - DNA, 2043 bp, Fusarium austroamericanum
atgaaacaccttttaacactcgctctttgcttcagcagcatcaatgctgttgctgtcaccgtccctcacaaggccgtaggaactggaattcctgaagggagtcttcagttcctgagccttcgagcctcagcacctatcggaagcgccatttctcgcaacaactgggccgtcacttgcgacagtgcacagtcgggaaatgaatgcaacaaggccattgatggcaacaaggataccttttggcacacattctatggcgccaacggggatccaaagccccctcacacatacacgattgacatgaagacaactcagaacgtcaacggcttgtctatgctgcctcgacaggatggtaaccaaaacggctggatcggtcgccatgaggtttatctaagctcagatggcacaaactggggcagccctgttgcgtcaggtagttggttcgccgactctactacaaaatactccaactttgaaactcgccctgctcgctatgttcgtcttgtcgctatcactgaagcgaatggccagccttggactagcattgcagagatcaacgtcttccaagctagttcttacacagccccccagcctggtcttggacgctggggtccgactattgacttaccgattgttcctgcggctgcagcaattgaaccgacatcgggacgagtccttatgtggtcttcatatcgcaatgatgcatttggaggatcccctggtggtatcactttgacgtcttcctgggatccatccactggtattgtttccgaccgcactgtgacagtcaccaagcatgatatgttctgccctggtatctccatggatggtaacggtcagatcgtagtcacaggtggcaacgatgccaagaagaccagtttgtatgattcatctagcgatagctggatcccgggacctgacatgcaagtggctcgtgggtatcagtcatcagctaccatgtcagacggtcgtgtttttaccattggaggctcctggagcggtggcgtatttgagaagaatggcgaagtctatagcccatcttcaaagacatggacgtccctacccaatgccaaggtcaacccaatgttgacggctgacaagcaaggattgtaccgttcagacaaccacgcgtggctctttggatggaagaagggttcggtgttccaagcgggacctagcacagccatgaactggtactataccagtggaagtggtgatgtgaagtcagccggaaaacgccagtctaaccgtggtgtagcccctgatgccatgtgcggaaacgctgtcatgtacgacgccgttaaaggaaagatcctgacctttggcggctccccagattatcaagactctgacgccacaaccaacgcccacatcatcaccctcggtgaacccggaacatctcccaacactgtctttgctagcaatgggttgtactttgcccgaacgtttcacacctctgttgttcttccagacggaagcacgtttattacaggaggccaacgacgtggaattccgttcgaggattcaaccccggtatttacacctgagatctacgtccctgaacaagacactttctacaagcagaaccccaactccattgttcgcgtctaccatagcatttcccttttgttacctgatggcagggtatttaacggtggtggtggtctttgtggcgattgtaccacgaatcatttcgacgcgcaaatctttacgccaaactatctttacaatagcaacggcaatctcgcgacacgtcccaagattaccagaacctctacacagagcgtcaaggtcggtggcagaattacaatctcgacggattcttcgattagcaaggcgtcgttgattcgctatggtacagcgacacacacggttaatactgaccagcgccgcattcccctgactctgacaaacaatggaggaaatagctattctttccaagttcctagcgactctggtgttgctttgcctggctactggatgttgttcgtgatgaactcggccggtgttcctagtgtggcttcgacgattcgcgttactcagtga

SEQ ID NO: 2 - protein, 680 aa, Fusarium austroamericanum
  mat_peptide (42)..(680)
MKHLLTLALCFSSINAVAVTVPHKAVGTGIPEGSLQFLSLRASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 3 - DNA, 2049 bp, Fusarium longipes
atgaaacagctcttgacacttgctctttgcttcagcagcatcaatgctgttgctataaaccctcatcaacatcacagcaacgagggaactggagatcatgaaggaagtctacagttcttgagtcttcgtgcatcagcacctttgggaagtgccatcgcccgcgacaactgggccgtgacttgtgatagtgcacagccaggaaatgagtgtaacaaagccattgatggcgacaccaataccttttggcatacattctatggaaacaatggtgacccaaagcctcctcatacttacaccattgacatgggatccaaccggaacgtcaatggattatctgttttacctcgccaggacggcaacagaaatggttggatcggcagacatgaggtttatctcagcactgatggcagcaactggggaagtcctgtcgcgtatggtagttggttcgccgattccacaaccaagtactccaacttcgaaactcgtcctgctcgctatgttcgtctagttgctcttactgaagcgagcggccagccttggaccagcatcgccgagattaatgtgtaccaggccggttcttacacagcccctcaggcgggtcttggacgatggggtccaactattgacttgcccatcgttccagcggctgctgcaatcgagccgacatcgggaagagtcctcatgtggtcgtcgtatcgtaacgatgcatttggaggctcccctggcggtatcactttgacatcgtcctgggatccatccagcggaattgtttcggaccgtactgtgactgtcaccaatcatgatatgttctgccccggtatctccatggatggtaatggtcagattgtggtcacgggcggaaacgacgcgaagaagacgagtctttatgactcgtctagtgacagctggatcccaggacctgacatgcaagttgctcgtggatatcagtcgtcggctacaatgtcagacggcagagtctttactataggtggatcctggagcggcggtatctttgagaagaatggcgaagtttatagtccatcttcaaagacatggacttccctccccggtgccaaggtcaacccaatgttgactgccgacaagcaaggtctgtaccgttcagacaaccacgcatggctctttggatggaagaaaggatcagttttccaagccggacccagcactgccatgaactggtactacaccagcggaagcggtgatgtaaagtcagccggaagacgtcagtccaaccgtggactagaccctgatgccatgtgcggaaatgctgtcatgtacgacgcagtcgctggcaagatcctgacgtttggtggctccccagactatcaagactctgacgccacaaccaacgcccacatcatcaccctcggtgaacccgggtccactcccaatactgtatttgccagcaacggtttatactttccccgaacctttcatacctctgtcgttctccctgacggaagcacgtttattaccggaggccaacgacgtggaattccgtttgaggattcgacccctcagcttacgcccgagatctatgtccctgagcaagacaccttttacaaacaaaaccccaactctattgttcgcgtctatcacagtatttccctattgttgcctgacggtagagtcttcaacggtggcggtggcctttgcggcgattgtaccacgaaccactttgacgcccagatctttacaccaaactatctttacaacagcaacggcgatcttgctacgcgtcccaagatcactagaacttctgcgcagagcgtcagggttggtggcaggattaccatctcgacggattcttcgattagaagagcgtctttgattcgctatggaacagcgacacatacagttaatactgaccaacgccgcattcctctgacgctgacgaacaacggaggaaacagttattctttccaagttcctagcgactctggtattgctttgcctggttactggatgttgtttgtgatgaactcggcgggcgtgccgagtgtagcaacgactcttcgcgttactcgatag

SEQ ID NO: 4 - protein, 682 aa, Fusarium longipes
  mat_peptide (44)..(682)
MKQLLTLALCFSSINAVAINPHQHHSNEGTGDHEGSLQFLSLRASAPLGSAIARDNWAVTCDSAQPGNECNKAIDGDTNTFWHTFYGNNGDPKPPHTYTIDMGSNRNVNGLSVLPRQDGNRNGWIGRHEVYLSTDGSNWGSPVAYGSWFADSTTKYSNFETRPARYVRLVALTEASGQPWTSIAEINVYQAGSYTAPQAGLGRWGPTIDLPIVPAAAAIEPTSGRVLMWSSYRNDAFGGSPGGITLTSSWDPSSGIVSDRTVTVTNHDMFCPGISMDGNGQIVVTGGNDAKKTSLYDSSSDSWIPGPDMQVARGYQSSATMSDGRVFTIGGSWSGGIFEKNGEVYSPSSKTWTSLPGAKVNPMLTADKQGLYRSDNHAWLFGWKKGSVFQAGPSTAMNWYYTSGSGDVKSAGRRQSNRGLDPDAMCGNAVMYDAVAGKILTFGGSPDYQDSDATTNAHIITLGEPGSTPNTVFASNGLYFPRTFHTSVVLPDGSTFITGGQRRGIPFEDSTPQLTPEIYVPEQDTFYKQNPNSIVRVYHSISLLLPDGRVFNGGGGLCGDCTTNHFDAQIFTPNYLYNSNGDLATRPKITRTSAQSVRVGGRITISTDSSIRRASLIRYGTATHTVNTDQRRIPLTLTNNGGNSYSFQVPSDSGIALPGYWMLFVMNSAGVPSVATTLRVTR

SEQ ID NO: 5 - DNA, 2043 bp, Fusarium austroamericanum
atgaaacaccttttaacactcgctctttgcttcagcagcatcaatgctgttgctgtcaccgtccctcacaaggccgtaggaactggaattcctgaagggagtcttcagttcctgagccttcgagcctcagcacctatcggaagcgccatttctcgcaacaactgggccgtcacttgcgacagtgcacagtcgggaaatgaatgcaacaaggccattgatggcaacaaggataccttttggcacacattctatggcgccaacggggatccaaagccccctcacacatacacgattgacatgaagacaactcagaacgtcaacggcttgtctatgctgcctcgacaggatggtaaccaaaacggctggatcggtcgccatgaggtttatctaagctcagatggcacaaactggggcagccctgttgcgtcaggtagttggttcgccgactctactacaaaatactccaactttgaaactcgccctgctcgctatgttcgtcttgtcgctatcactgaagcgaatggccagccttggactagcattgcagagatcaacgtcttccaagctagttcttacacagccccccagcctggtcttggacgctggggtccgactattgacttaccgattgttcctgcggctgcagcaattgaaccgacatcgggacgagtccttatgtggtcttcatatcgcaatgatgcatttggaggatcccctggtggtatcactttgacgtcttcctgggatccatccactggtattgtttccgaccgcactgtgacagtcaccaagcatgatatgttctgccctggtatctccatggatggtaacggtcagatcgtagtcacaggtggcaacgatgccaagaagaccagtttgtatgattcatctagcgatagctggatcccgggacctgacatgcaagtggctcgtgggtatcagtcatcagctaccatgtcagacggtcgtgtttttaccattggaggctcctggagcggtggcgtatttgagaagaatggcgaagtctatagcccatcttcaaagacatggacgtccctacccaatgccaaggtcaacccaatgttgacggctgacaaggaaggattgaagaagtcagacaaccacgcgtggctctttggatggaagaagggttcggtgttccaagcgggacctagcacagccatgaactggtactataccagtggaagtggtgatgtgaagtcagccggaaaacgccagtctaaccgtggtgtagcccctgatgccatgtgcggaaacgctgtcatgtacgacgccgttaaaggaaagatcctgacctttggcggctccccagattatcaagactctgacgccacaaccaacgcccacatcatcaccctcggtgaacccggaacatctcccaacactgtctttgctagcaatgggttgtactttgcccgaacgtttcacacctctgttgttcttccagacggaagcacgtttattacaggaggccaacgacgtggaattccgttcgaggattcaaccccggtatttacacctgagatctacgtccctgaacaagacactttctacaagcagaaccccaactccattgttcgcgtctaccatagcatttcccttttgttacctgatggcagggtatttaacggtggtggtggtctttgtggcgattgtaccacgaatcatttcgacgcgcaaatctttacgccaaactatctttacaatagcaacggcaatctcgcgacacgtcccaagattaccagaacctctacacagagcgtcaaggtcggtggcagaattacaatctcgacggattcttcgattagcaaggcgtcgttgattcgctatggtacagcgacacacacggttaatactgaccagcgccgcattcccctgactctgacaaacaatggaggaaatagctattctttccaagttcctagcgactctggtgttgctttgcctggctactggatgttgttcgtgatgaactcggccggtgttcctagtgtggcttcgacgattcgcgttactcagtga

SEQ ID NO: 6 - protein, 680 aa, Fusarium austroamericanum
  mat_peptide (42)..(680)
MKHLLTLALCFSSINAVAVTVPHKAVGTGIPEGSLQFLSLRASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKEGLKKSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 7 - DNA, 2043 bp, Fusarium austroamericanum
atgaaacacctcctcacattggcgctctgtttctcgtcgatcaacgcggtcgccgtcaccgtcccccacaaggccgtcggcaccggcatccctgagggctccttgcagttcctctcgctcagggcgtccgcacctatcggttcggcgatctccaggaacaactgggcagtcacttgtgactcggcacagtccggtaacgaatgtaacaaagcgattgatggaaacaaggacactttctggcataccttctatggcgcaaacggcgatccgaaacctccccatacttatactattgacatgaaaaccacgcagaacgtgaacggcttgtcgatgttgcctcgacaggacggcaaccagaacggctggattggtcgacacgaggtctacctctcgtccgacggaaccaactggggctcgcctgtcgcatcgggctcctggttcgcagactccacgacgaagtactccaacttcgagactaggcctgcacgatacgtcaggctcgtggcaattacagaagccaacggccagccgtggacatcgatcgcagaaatcaacgtcttccaggcctcgtcctacacagcccctcagcctggcctcggacgttggggtcccacgatcgacttgcccatcgtgcctgcagcggcagccatcgaacctacatcgggacgggtcttgatgtggtcctcgtacaggaacgacgccttcggtggatcgcctggaggcatcaccctcacctcctcctgggacccctcgaccggcatcgtgtcggataggactgtcactgtcactaagcacgacatgttctgtcctggaatctcgatggatggcaacggccagatcgtcgtcaccggtggtaacgacgcgaaaaagacctcgctctatgattcgtcctccgattcctggattcctggaccggatatgcaggtcgcaaggggctaccagtcgtccgccacaatgtccgacggcagggtcttcacaattggaggctcgtggtcgggtggagtgttcgagaaaaacggcgaagtctactcgccttcctcgaagacatggacatcgctccccaacgccaaagtcaaccccatgctcacagcagacaaagagggcctcaagaagtccgacaaccacgcctggttgttcggatggaagaaaggctcggtgttccaggcaggtccctccactgccatgaactggtactacacctcgggttcgggagatgtcaaatccgcaggaaaacggcagtccaaccgaggcgtcgcaccggacgcgatgtgtggtaacgccgtgatgtacgacgccgtcaagggcaaaatcctcaccttcggtggctcccctgactacacggattcggacgcgactaccaacgcacatatcattaccctcggagaacctggcacctcccccaacaccgtgttcgcatccaacggtctctacttcgccaggaccttccacacgtccgtcgtcctcccggatggctccaccttcatcacaggcggtcagaggcgaggtatcccgttcgaggattccacaccggtcttcactcccgagatttacgtccccgagcaggatacgttctacaaacagaacccgaactccatcgtcagggtctaccactccatctcgctcttgctccccgatggccgagtcttcaacggaggaggaggcttgtgtggtgattgtaccacgaaccacttcgacgcgcagatcttcacccccaactatttgtacaactccaacggtaacttggcaacgaggccgaagatcacaaggacgtcgacacagtcggtcaaggtcggaggccgaattacaatctcgacggactcctccatctccaaggcatccttgattcggtacggtacagcaacgcataccgtgaacaccgatcagaggcgaatccccctcaccttgaccaacaacggaggtaactcctattccttccaggtgccgtccgattccggagtcgcgctccctggctactggatgctcttcgtgatgaactccgcaggagtgccctccgtcgcatccacaattagggtcacccagtga

SEQ ID NO: 8 - protein, 680 aa, Fusarium austroamericanum
  mat_peptide (42)..(680)
MKHLLTLALCFSSINAVAVTVPHKAVGTGIPEGSLQFLSLRASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKEGLKKSDNHAWLFGWKKGSVFQAGPSTAMNWYYTSGSGDVKSAGKRQSNRGVAPDAMCGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 9 - protein, 328 aa, Agrocybe aegerita
EPGLPPGPLENSSAKLVNDEAHPWKPLRPGDIRGPCPGLNTLASHGYLPRNGVATPVQIINAVQEGLNFDNQAAVFATYAAHLVDGNLITDLLSIGRKTRLTGPDPPPPASVGGLNEHGTFEGDASMTRGDAFFGNNHDFNETLFEQLVDYSNRFGGGKYNLTVAGELRFKRIQDSIATNPNFSFVDFRFFTAYGETTFPANLFVDGRRDDGQLDMDAARSFFQFSRMPDDFFRAPSPRSGTGVEVVIQAHPMQPGRNVGKINSYTVDPTSSDFSTPCLMYEKFVNITVKSLYPNPTVHVRKALNTNLDFFFQGVAAGCTQVFPYGRD

SEQ ID NO: 10 - protein, 344 aa, Coprinopsis cinerea
TSKLPIVFPPPPPEPIKDPWLKLVNDRAHPWRPLRRGDVRGPCPGLNTLASHGYLPRDGVATPAQIITAVQEGFNMEYGIATFVTYAAHLVDGNPLTNLISIGGKTRKTGPDPPPPAIVGGLNTHAVFEGDASMTRGDFHLGDNFNFNQTLWEQFKDYSNRYGGGRYNLTAAAELRWARIQQSMATNGQFDFTSPRYFTAYAESVFPINFFTDGRLFTSNTTAPGPDMDSALSFFRDHRYPKDFHRAPVPSGARGLDVVAAAYPIQPGYNADGKVNNYVLDPTSADFTKFCLLYENFVLKTVKGLYPNPKGFLRKALETNLEYFYQSFPGSGGCPQVFPWGKSD

SEQ ID NO: 11 - protein, 351 aa, Coprinopsis cinerea
MVSCKLPLPLLTLAIALANVNAFPAYQSLGGLSKRQLETIIPGLPVVNPGPPPGPLADSTLKLVNDAAHPYQAPRPHLDHRGPCPGLNTLANHGYLPRSGIATPAQIVQAVMEGFNMENTFAKFVTYAAFLVDGNPITNLMSIGGKTWRTGIIEPPPPAIVGGLNTHAVFEGDTSMTRGDFHFGDNHSFNQTLFDQFVEYSNIHGGGFYNLTAATELRYQRIQQSIATNPEMSFVSPRWFTAILLQDEKFPDDFHRAPGPFSFEGLGYLVTRRPMPPGRNVGGVDNYVPDPNSADFNSFCKMYEDFVNDIVVALYPNPTGLLRRNLIKNLEYFWTGMFDPACTEVKPYGTL

SEQ ID NO: 12 - protein, 243 aa, Aspergillus niger
LATGSTKLLPWSPPGHGDVRGPCPMLNTLANHGLLPHNGKDISQEVITEVLNNTLNLADGLSAFLFEEAMTTVEDPKATTFSLSDLNCPGILEHDGSLSRQDTYFGNNHEFNQTIFDQTKSYWTTPLIDMYQAAEAHEARLNTSKATNPTFNLSETGLTFSFGETAAYMIVFEDTNLGYANRSWVEYFFENERLPQELGWTKRPFITTGQVLVDMTTWVINSTIGVTPEEQAEMQDFGKKITG

SEQ ID NO: 13 - protein, 259 aa, Aspergillus niger
LATGSTKLLPWSPPGHGDVRGPCPMLNTLANHGLLPHNGKDISQEVITEVLNNTLNLADGLSAFLFEEAMTTVEDPKATTFSLSDLNCPGILEHDGSLRYLPLQKHRGNQADLLSRQDTYFGNNHEFNQTIFDQTKSYWTTPLIDMYQAAEAHEARLNTSKATNPTFNLSETGLTFSFGETAAYMIVFEDTNLGYANRSWVEYFFENERLPQELGWTKRPFITTGQVLVDMTTWVINSTIGVTPEEQAEMQDFGKKITG

SEQ ID NO: 14 - protein, 247 aa, Aspergillus niger
FPQQGAPHPLPWSPPGPNDVRAPCPMLNTLANHGYLPHNGKDITERHTINALYNALGIEEELAIYLHQEAVTTNPAPNATTFSLNDLSRHDILEHDASLSRQDAYFGDNHDFNQTIFDETRSYWTSPIIDVKQAAVSRQARVNTSMATNPNYTMSELGDSFSYGETAAYIIVLGDKEKGLVNRSRVEYLFENERLPLDLGWSRAKENITFDDLSTMLQRIINATGGEMDFRATIALPRLVYIYYEEA

SEQ ID NO: 15 - protein, 247 aa, Aspergillus niger
FPQQGAPHPLPWSPPGPNDVRAPCPMLNTLANHGYLPHNGKDITERHTINALYNALGIEEELAIYLHQEAVTTNPAPNATTFSLNDLSRHDILEHDASLSRQDAYFGDNHDFNQTIFDETRSYWTSPIIDVKQAAVSRQARVNTSMATNPNYTMSELGDSFSYGETAAYIIVLGDKEKGLVNRSRVEYLFENERLPLDLGWSRAKENITFDDLSTMLQRIINATGGESEFDRELAKRGGVHVGSWRG

SEQ ID NO: 16 - protein, 259 aa, Poronia punctata
KAACPYGYGEFQPEQTSDARGPCPVLNTLANHGYLPRDGRHIDENRTLTALHDALNLDIDFGKFLFTAGRLSNPKANSTWFDLDHLSRHGIFEHDGSLSRQDHHFGEWSRFNQTVWNWTLEYLPDDMLDVQTVANARAQRMTRSNLTNPDFALSYLGYLFSVGEAAAVLSILGDKKTQTCPKAFADYIFVNERLPYELGWKKQDASISFDDLVETFEDLERHTSFPFPPPLDNSTDIFDQLVEGGQSKKKRCSAHIGCF

SEQ ID NO: 17 - protein, 259 aa, Chaetomium virescens
ELDFSKWKTRQPGEFRAPCPAMNSLANHGFIPRDGRNITVAMLVPVLQEVFHLSPELAQTISTLGLFTAQDPSKGVFTLDDLNRHNLFEHDASLSREDYYFHKDASTFRPEVFKKFMSHFKGKEYVTLEDAASARYAMVQESRKKNPTFTYTVQQRITSYGETIKYFRTIVEPATGKCPVAWIKILFEQERLPYNEGWRPPKAELSGFSMASDVLELALVTPEKLIDKPCEGKQCPQARGIHGYFGMLLPITAQELAVK

SEQ ID NO: 18 - protein, 247 aa, Chaetomium virescens
ELDFSKWKTRQPGEFRAPCPAMNSLANHGFIPRDGRNITVAMLVPVLQEVFHLSPELAQTISTLGLFTAQDPSKGVFTLDDLNRHNLFEHDASLSREDYYFHKDASTFRPEVFKKFMSHFKGKEYVTLEDAASARYAMVQESRKKNPTFTYTVQQRITSYGETIKYFRTIVEPATGKCPVAWIKILFEQERLPYNEGWRPPKAELSGFSMASDVLELALVTPEKLIDKPCEGKQCPQARGIHGYFGM

SEQ ID NO: 19 - protein, 261 aa, Humicola insolens
GFHDWEPPGPNDVRAPCPMLNTLANHGFLPHHGRDLTRKQVVDGLYNGLNINKTAASTLFDFALMTSPKPNATTFSLDDLGRHNILEHDASLSRTDAYFGDVLAFNKTIFEETKRHWGKSPILDVTAAARARLGRIQTSKATNPEYFMSELGNIFTYGESVAYIMLIGDAKTGRANRRWVEYWFENERLPTHLGWRRPSKELTSDVLDTYISLIQNITLTLPGGTDPVKRRAASHFVFPFGQGLGGPAGVALMLISVVAYG

SEQ ID NO: 20 - protein, 238 aa, Humicola insolens
GFHDWEPPGPNDVRAPCPMLNTLANHGFLPHHGRDLTRKQVVDGLYNGLNINKTAASTLFDFALMTSPKPNATTFSLDDLGRHNILEHDASLSRTDAYFGDVLAFNKTIFEETKRHWGKSPILDVTAAARARLGRIQTSKATNPEYFMSELGNIFTYGESVAYIMLIGDAKTGRANRRWVEYWFENERLPTHLGWRRPSKELTSDVLDTYISLIQNITLTLPGGTDPVKRRAASHFGW

SEQ ID NO: 21 - protein, 242 aa, Chaetomium globosum
GFDTWAPPGPYDVRGPCPMLNTLTNHGFFPHDGQDIDRETTENALFDALHVNKTLASFLFDFALTTNPIANSTTFSLNDLGNHNVLEHDASLSRADAYHGSVLAFNHTIFEETKSYWTDETVTLKMAADARYYRIKSSQATNPTYQMSELGDAFTYGESAAYVVLFGDKESQTVPRSWVEWLFEKEQLPQHLGWKRPATSFELNDLDKFMALIQNYTQEIEEPSCESRKQRRKPRGPSHFGF

SEQ ID NO: 22 - protein, 301 aa, Chaetomium globosum
GFDTWAPPGPYDVRAPCPMLNTLANHGFLPHDGHEITREQTENALFDALHIDKMLGSSLFDFAMTTNPVANSTTFSLNDLGNHNVLEHDASLSRSDAYFGNTLTFNQTVFDETKSYWTDETVTIEMASNARLARIKTSNATNPTYSMSELGNGFTKGESAAYVVIFGDKISGTVPRAWVEWLFEHEQLPQHLGWKRPTELFRDGDLDKYMDAMQNVIVEIALKTQPSTPSIKPTQTPSSPTRLLLKRLGRQLMLIVPRPIRLRVLRNTPPLRLITKNKPREMAPNLLILAVHKRATSMQKR

SEQ ID NO: 23 - protein, 344 aa, Chaetomium globosum
GFDTWAPPGPYDVRAPCPMLNTLANHGFLPHDGHEITREQTENALFDALHIDKMLGSSLFDFAMTTNPVANSTTFSLNDLGNHNVLEHDASLSRSDAYFGNTLTFNQTVFDETKSYWTDETVTIEMASNARLARIKTSNATNPTYSMSELGNGFTKGESAAYVVIFGDKISGTVPRAWVEWLFEHEQLPQHLGWKRPTELFRDGDLDKYMDAMQNVIVGETPGCPAGKQQQRKGRRTPSHFGWDPTPVNKLASLRIAGYCHEIALKTQPSTPSIKPTQTPSSPTRLLLKRLGRQLMLIVPRPIRLRVLRNTPPLRLITKNKPREMAPNLLILAVHKRATSMQKR

SEQ ID NO: 24 - protein, 243 aa, Chaetomium globosum
GFDTWAPPGPYDVRAPCPMLNTLANHGFLPHDGHEITREQTENALFDALHIDKMLGSSLFDFAMTTNPVANSTTFSLNDLGNHNVLEHDASLSRSDAYFGNTLTFNQTVFDETKSYWTDETVTIEMASNARLARIKTSNATNPTYSMSELGNGFTKGESAAYVVIFGDKISGTVPRAWVEWLFEHEQLPQHLGWKRPTELFRDGDLDKYMDAMQNVIVGETPGCPAGKQQQRKGRRTPSHFGW

SEQ ID NO: 25 - protein, 276 aa, Sclerotinia sclerotiorum
MKLNFLSTTLALGLVSARAHYQQQIIANDTEGEWIAPSATDYRGPCPMLNTLANHGFLPRDGRNLTEHNVVKGLNHGLNFNKSLGSIMFQHAVPASPAYPNTTFFTLDDLNRHNVLEHDASISRSDAYFGNNHIFNQTIFDTTKMYWPSETLTAQHLIDGKIFRQIVSRTTNPNYTFTSTTQAFSLGEMAAPIVAFGDKNALTANRTLVESWIENERLPTELGWSKPEEEVSLGDILYVTGALANLTSLLSDVVITPRGESAGAHAKRMGHWGVSM

SEQ ID NO: 26 - protein, 259 aa, Aspergillus carbonarius
MKSTILLITTSLSQALAQVSSHPFPWSAPGPNDVRGPCPMLNTLANHGFLPHDGKDITEDRIVMVLNNSLNLDEELSQFLFKEALTTNPDPNATTFSLNDLSRHNILEHDASLSRQDYYFGDNHDFNQTVFNETRSYWTAPLIDFNAAAQARLARVNTSMATNPTYTESETGLAFSYGESAAYMIVFAEGSETANRSWVEYFFEHERLPQQLGWTKPQESISSSVLIDTVTGIANASNASSLVVAELLDFVALHMGRLP

SEQ ID NO: 27 - protein, 8 aa, Artificial Sequence
  MOTIF SEQUENCE
EHDXSXSR

SEQ ID NO: 28 - protein, 259 aa, Chaetomium virescens; identical to SEQ ID NO: 17

SEQ ID NO: 29 - protein, 244 aa, Daldinia caldariorum
APWKAPGPDDVRGPCPMLNTLANHGFLPHDGKNIDVNTTVNALSSALNLDDELSRDLHTFAVTTNPQPNATWFSLNHLSRHNVLEHDASLSRQDAYFGPPDVFNAAVFNETKAYWTGDIINFQMAANALTARLMTSNLTNPEFSMSQLGRGFGLGETVAYVTILGSKETRTVPKAFVEYLFENERLPYELGFKKMKSALTEDELTTMMGEIYSLQHLPESFTKPFAKRSEAPFEKRAEKRCPFH

SEQ ID NO: 30 - protein, 244 aa, Myceliophthora fergusii
GFDTWSPPGPYDVRAPCPMLNTLANHGFLPHDGKDITREQTENALFEALHINKTLGSFLFDFALTTNPRNTSTFSLNDLGNHNILEHDASLSRADAYFGNVLQFNQTVFDETKTYWDGDVIDLRMAARARLGRIKTSQATNPTYSMSELGDAFTYGESAAYVVVLGDKESRTAKRSWVEWFFEHEQLPQHLGWKRPASSLEEEDLFTIMDEIRQYTSELEGSTSSSDAQTSRRQLPRRRTHFGF

SEQ ID NO: 31 - protein, 244 aa, Myceliophthora hinnulea
GFDTWSPPGPYDVRAPCPMLNTLANHGFLPHDGKDITREQTENALFDALNINKTLASFLFDFALTTNPKNTSTFSLNDLGNHNILEHDASLSRADAYFGNVLQFNQTVFDETKTYWEGDTIDLRMAAKARLGRIKTSQATNPTYSMSELGDAFTYGESAAYVVVLGDKESRTVNRSWVEWFFEHEQLPQHLGWKRPAVSFEEEDLNRFMEEIEKYTKGLEGSNSTSGSQKHRRRLPRRRTHFGF

SEQ ID NO: 32 - protein, 241 aa, Thielavia hyrcaniae
GFDTWSPPGPYDVRAPCPMLNTLANHGFLPHDGKDLTRDVVENALSDALNINKTLGSFLFDFALTTNPKPNSTTFSLNDLGNHNILEHDASLSRSDAYFGNVLVFNQSVFDETKSYFKGKTVTLKQAAQARLARIKTSKATNPTYSMSQLGDSFTYGESAAYVVVLGGDKKSATVPRSWVEWFFEHEQLPQHLGWKRPAEQFTQDDLNKFMDEIINITKAIDHRSVDVPEKTKRRISHWGA

SEQ ID NO: 33 - DNA, 54 bp, Fusarium longipes
acacaactggggatccaccatgaaacagctcttgacacttgctctttgcttcag

SEQ ID NO: 34 - DNA, 47 bp, Fusarium longipes
agatctcgagaagcttatcgagtaacgcgaagagtcgttgctacact

(19) United States
(12) Patent Application Publication
(10) Pub. No.: US 2015/0152452 A1
(43) Pub. Date: Jun. 4, 2015

(54) Enzymatic Oxidation of 5-Hydroxymethylfurfural and Derivatives Thereof

(71) Applicant: Novozymes A/S, Bagsvaerd (DK)

(72) Inventors: Lisbeth Kalum, Vaerloese (DK); Marc Dominique Morant, Copenhagen (DK); Henrik Lund, Vaerloese (DK); Jacob Jensen, Bagsvaerd (DK); Ingrida Lapainaite, Bagsvaerd (DK); Niels Henrik Soerensen, Skaevinge (DK); Sven Pedersen, Gentofte (DK); Lars Henrik Ostergaard, Charlottenlund (DK); Feng Xu, Davis (CA)

(21) Appl. No.: 14/414,251

(22) PCT Filed: Jul. 19, 2013
(86) PCT No.: PCT/US2013/051272

§ 371 Date: Jan. 12, 2015

Related U.S. Application Data
(60) Provisional application No. 61/673,913, filed on Jul. 20, 2012.

(51) Int. Cl. C12P 17/04

(52) U.S. Cl. CPC C12P 17/04

(57) ABSTRACT

Provided herein are enzymatic methods for oxidation of 5-hydroxymethylfurfural (HMF) and HMF derivatives.

### CROSS-REFERENCE TO RELATED APPLICATIONS

This application claims priority from U.S. provisional application Ser. No. 61/673,913 filed Jul. 20, 2012. The content of this application is fully incorporated herein by reference.

### REFERENCE TO A SEQUENCE LISTING

This application contains a Sequence Listing in computer readable form, which is incorporated herein by reference.

### FIELD OF THE INVENTION

The present invention relates to processes for oxidizing 5-hydroxymethylfurfural (HMF), 2,5-diformylfuran (DFF), 5-hydroxymethyl-2-furancarboxylic acid (HMFCA), and formylfuran carboxylic acid (FFCA) by catalytic oxidation with galactose oxidase and/or peroxygenase.

### BACKGROUND

Chemical compounds needed for various industries have for many years been derived from the petrochemical industry. However, due to increases in the price of crude oil and a general awareness of replacing petrochemicals with renewable resources there has been and still is a wish to base the production of chemical compounds on renewable resources.

5-hydroxymethylfurfural (HMF; CAS: 67-47-0) is an example of such a compound because it is derived from dehydration of sugars making it obtainable from renewable resources. HMF can for example be converted to a variety of useful products, such as the liquid biofuel 2,5-dimethylfuran by hydrogenolysis of C—O bonds over a copper-ruthenium (CuRu) catalyst (Roman-Leshkov Y et al. Nature 2007, 447, 982), or to 2,5-furan dicarboxylic acid (FDCA) by oxidation (Boisen A et al., Chemical Engineering Research and Design, 2009, 87, 1318-1327). The latter compound, FDCA, can be used as a replacement of terephthalic acid in the production of polyesters such as polyethyleneterephthalate (PET) and polybutyleneterephthalate (PBT). One drawback of FDCA is that the chemical synthesis requires high pressure, high temperature, metal salts and organic solvents, rendering the process expensive and polluting (Koopman et al. Bioresource Technology 2010, 101, 6291-6296).

2,5-diformylfuran (DFF; CAS: 823-82-5) is an oxidized dialdehyde of HMF that can be used a building block and cross-linking agent in a range of different applications. For example, DFF can be used as a monomer for polymer production, e.g., in combination with urea, or can be further oxidized to useful building blocks such as FFCA and FDCA. It can also replace other aldehydes commonly used, such as glutaraldehyde for cross-linking of leather or formaldehyde for cross-linking of wood composites in combination with urea, melamin and/or phenol. However, selective oxidation of HMF to DFF by traditional chemical methods is difficult because the reaction often indiscriminately oxidizes resulting in a combination of oxidation products.

The selective oxidation of HMF by enzymatic catalysis may provide an alternative to chemical methods due to heightened enzyme-substrate specificity. However, HMF is not a known natural enzyme substrate so identifying enzymes with a suitable structure capable of selectively oxidizing HMF would be challenging.

Deurzen et al., J Carbohydrate Chemistry 1997, 16, 299-309 describes the oxidation of HMF to DFF with hydrogen peroxide using chloroperoxidase catalyst. WO2009/023174 demonstrates the oxidation of HMF to DFF and other HMF oxidation products using, e.g., aryl alcohol oxidase and chloroperoxidase enzymes. WO2008/119780 demonstrates the use of fungal peroxygenases to generate N-oxides from pyridine.

However, due to the variability in enzymatic properties (e.g., stability and activity under varying conditions) it would be advantageous in the art to identify alternative enzymes capable of producing products of HMF oxidation, such as DFF, FFCA and FDCA. The present invention provides, inter alia, methods for making such oxidized products.

### SUMMARY

Described herein are enzymatic methods of oxidizing 5-hydroxymethylfurfural (HMF) and HMF derivatives using galactose oxidase and/or peroxygenase.

In one aspect is a method of oxidizing 5-hydroxymethylfurfural (HMF), comprising contacting HMF with a galactose oxidase in a reaction mixture under suitable conditions to provide 2,5-diformylfuran (DFF). In some embodiments, the galactose oxidase has at least 60% sequence identity to the mature polypeptide sequence of SEQ ID NO: 2. In some embodiments, the galactose oxidase is a variant comprising a substitution at one or more (several) positions corresponding to positions 326, 329, 330, and 406 of SEQ ID NO: 2. In embodiments, the reaction mixture further comprises a peroxygenase, and DFF is further oxidized to formylfuran carboxylic acid (FFCA), 2,5-furan dicarboxylic acid (FDCA), a salt thereof, or a mixture of the foregoing. In some of these embodiments, the peroxygenase has at least 60% sequence identity to the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32. In some embodiments, the peroxygenase is a variant comprising a substitution at one or more (several) positions corresponding to positions 76, 134, and 201 of SEQ ID NO: 10.

In one aspect is a method of oxidizing HMF, comprising contacting HMF with a peroxygenase in a reaction mixture under suitable conditions to provide DFF, HMFCA, FFCA, FDCA, a salt thereof, or a mixture of the foregoing. In some embodiments, the peroxygenase has at least 60% sequence identity to the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32. In some embodiments, the peroxygenase is a variant comprising a substitution at one or more (several) positions corresponding to positions 76, 134, and 201 of SEQ ID NO: 10.

In one aspect is a method of oxidizing DFF, comprising contacting DFF with a peroxygenase in a reaction mixture under suitable conditions to provide FFCA, FDCA, a salt thereof, or a mixture of the foregoing. In some embodiments, the peroxygenase has at least 60% sequence identity to the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32. In some embodiments, the peroxygenase is a variant comprising a substitution at one or more (several) positions corresponding to positions 76, 134, and 201 of SEQ ID NO: 10.

In one aspect is a method of oxidizing 5-hydroxymethyl-2-furancarboxylic acid (HMFCA) or a salt thereof, comprising contacting HMFCA or a salt thereof with a galactose oxidase in a reaction mixture under suitable conditions to provide FFCA or a salt thereof. In some embodiments, the galactose oxidase has at least 60% sequence identity to the mature polypeptide sequence of SEQ ID NO: 2. In some embodiments, the galactose oxidase is a variant comprising a substitution at one or more (several) positions corresponding to positions 326, 329, 330, and 406 of SEQ ID NO: 2. In some embodiments, the reaction mixture further comprises a peroxygenase. In some of these embodiments, FFCA is further oxidized to FDCA or a salt thereof. In some of these embodiments, the peroxygenase has at least 60% sequence identity to the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32. In some of these embodiments, the peroxygenase is a variant comprising a substitution at one or more (several) positions corresponding to positions 76, 134, and 201 of SEQ ID NO: 10.

In one aspect is a method of oxidizing HMFCA or a salt thereof, comprising contacting HMFCA or a salt thereof with a peroxygenase in a reaction mixture under suitable conditions to provide formylfuran carboxylic acid FFCA, FDCA, a salt thereof, or a mixture of the foregoing. In some embodiments, the peroxygenase has at least 60% sequence identity to the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32. In some of these embodiments, the peroxygenase is a variant comprising a substitution at one or more (several) positions corresponding to positions 76, 134, and 201 of SEQ ID NO: 10.

In one aspect is a method of oxidizing FFCA or a salt thereof, comprising contacting FFCA or a salt thereof with a peroxygenase in a reaction mixture under suitable conditions to provide FDCA or a salt thereof. In some embodiments, the peroxygenase has at least 60% sequence identity to the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32. In some of these embodiments, the peroxygenase is a variant comprising a substitution at one or more (several) positions corresponding to positions 76, 134, and 201 of SEQ ID NO: 10.

### BRIEF DESCRIPTION OF THE FIGURES

FIG. 1 shows oxidation products of 5-hydroxymethylfurfural (HMF).

FIGS. 2A and 2B show an alignment of galactose oxidase sequences of F. austroamericanum (native, SEQ ID NO: 2), F. austroamericanum (MutA, SEQ ID NO: 6), F. austroamericanum (MutB, SEQ ID NO: 8), and F. longipes (native, SEQ ID NO: 4). The published mature polypeptide start site for the F. austroamericanum galactose oxidase is shown with a vertical arrow. Substituted residues of the variant F. austroamericanum sequences are shown in boldface.

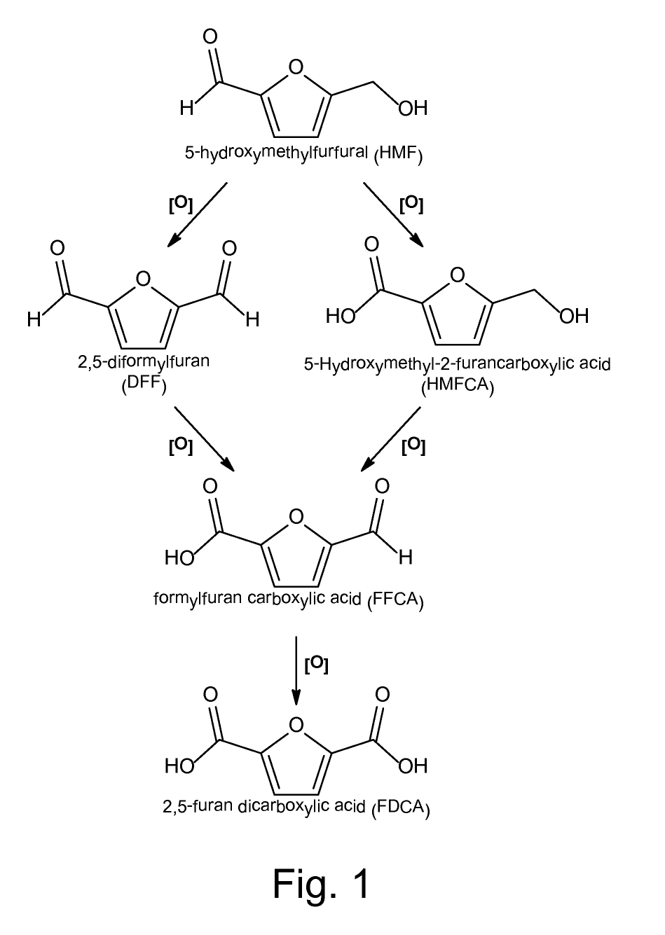

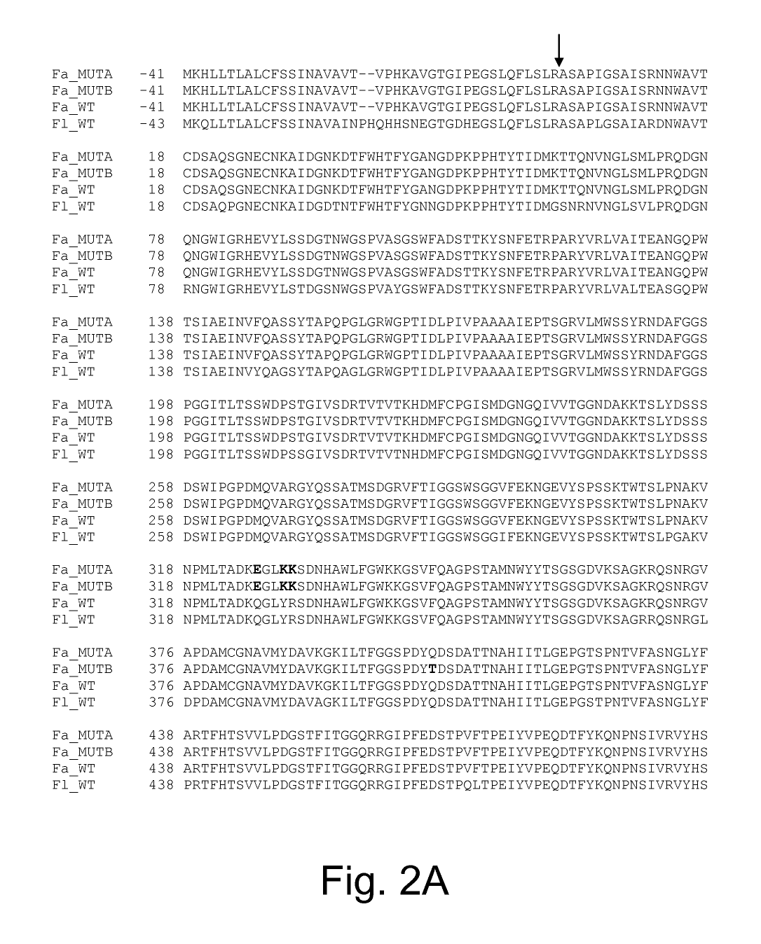

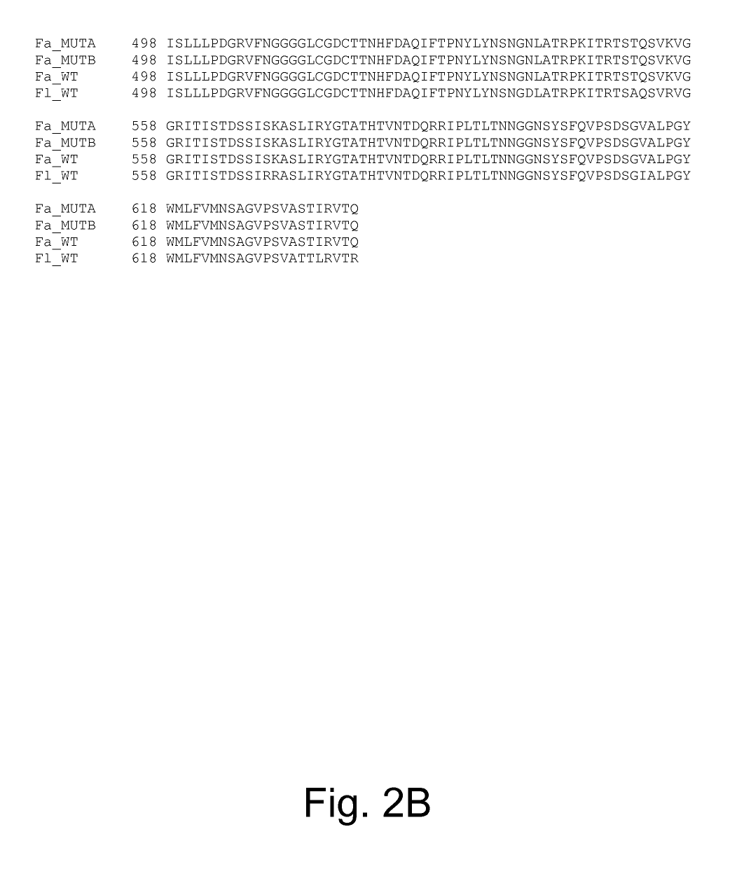

### DEFINITIONS

Galactose oxidase: The term “galactose oxidase” is defined herein as an oxidoreductase enzyme that catalyzes the conversion of D-galactose and oxygen to D-galactose-hexodialdose and H₂O₂ (EC 1.1.3.9). For purposes of the present invention, galactose oxidase activity may be determined according to the procedure described in Xu, F. et al. Appl Biochem Biotechnol 2000, 88, 23-32.

In some aspects, the galactose oxidase has at least 20%, e.g., at least 40%, at least 50%, at least 60%, at least 70%, at least 80%, at least 90%, at least 95%, at least 96%, at least 97%, at least 98%, at least 99%, or 100% of the galactose oxidase activity of the mature polypeptide sequence of SEQ ID NO: 2 under the same conditions.

Peroxygenase: The term “peroxygenase” means an “unspecific peroxygenase” activity according to EC 1.11.2.1, that catalyzes insertion of an oxygen atom from H₂O₂ into a variety of substrates, such as nitrobenzodioxole. For purposes of the present invention, peroxygenase activity may be determined according to the procedure described in Poraj-Kobielska, M. et al. Analytical Biochemistry 2012, 421, 327-329.

In some aspects, the peroxygenase has at least 20%, e.g., at least 40%, at least 50%, at least 60%, at least 70%, at least 80%, at least 90%, at least 95%, at least 96%, at least 97%, at least 98%, at least 99%, or 100% of the peroxygenase activity of the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32 under the same conditions.

Heterologous polynucleotide: The term “heterologous polynucleotide” is defined herein as a polynucleotide that is not native to the host cell; a native polynucleotide in which one or more (e.g., two, several) structural modifications have been made to the coding region; a native polynucleotide whose expression is quantitatively altered as a result of manipulation of the DNA by recombinant DNA techniques, e.g., a different (foreign) promoter linked to the polynucleotide; or a native polynucleotide whose expression is quantitatively altered by the introduction of one or more extra copies of the polynucleotide into the host cell.

Coding sequence: The term “coding sequence” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

cDNA sequence: The term “cDNA sequence” means a sequence of DNA following reverse transcription from a mature, spliced, mRNA molecule obtained from a eukaryotic cell. The initial, primary RNA transcript from genomic DNA is a precursor to mRNA that is processed through a series of steps, including splicing, before appearing as mature spliced mRNA. A cDNA sequence lacks intervening intron sequences that may be present in the corresponding genomic DNA sequence. Accordingly, the phrase “the cDNA sequence of SEQ ID NO: X” intends the resulting sequence after the intervening intron sequences of SEQ ID NO: X, if present, are removed. In some instances—when a referenced genomic DNA sequence lacks intervening intron sequences—a cDNA sequence may be identical to its corresponding genomic DNA sequence.

Genomic DNA sequence: The term “genomic DNA sequence” means a DNA sequence found in the genome of a source organism (e.g., a eukaryotic or prokaryotic genome). In some instances, a genomic DNA sequence from a eukaryotic genome contains one or more intervening intron sequences that are removed from the primary RNA transcript as a result of RNA splicing. Accordingly, the phrase “the genomic DNA sequence of SEQ ID NO: Y” intends the corresponding DNA sequence from the source organism which includes intervening intron sequences, if any, that are present before RNA splicing.

Mature polypeptide sequence: The term “mature polypeptide sequence” means the portion of the referenced polypeptide sequence after any post-translational sequence modifications (such as N-terminal processing and/or C-terminal truncation). The mature polypeptide sequence may be predicted, e.g., based on the SignalP program (Nielsen et al., 1997, Protein Engineering 10: 1-6) or the InterProScan program (The European Bioinformatics Institute). It is known in the art that a host cell may produce a mixture of two of more different mature polypeptide sequences (i.e., with a different C-terminal and/or N-terminal amino acid) expressed by the same polynucleotide.

In one aspect, the mature polypeptide of the galactose oxidase is amino acids 1 to 639 of SEQ ID NO: 2, 4, 6, or 8. In another aspect, the mature polypeptide of the galactose oxidase is amino acids 3 to 639 of SEQ ID NO: 2, 4, 6, or 8 (e.g., when recombinantly expressed by A. oryzae as described in Xu, F. et al. Appl Biochem Biotechnol 2000, 88, 23-32).

Mature polypeptide coding sequence: The term “mature polypeptide coding sequence” means the portion of the referenced polynucleotide sequence (e.g., genomic or cDNA sequence) that encodes a mature polypeptide sequence. The mature polypeptide coding sequence may be predicted, e.g., based on the SignalP program (supra) or the InterProScan program (supra). In some instances, the mature polypeptide coding sequence may be identical to the entire referenced polynucleotide sequence.

In one aspect, the mature polypeptide coding sequence of the galactose oxidase is nucleotides 124 to 2040 of SEQ ID NO: 1, 5, or 7, or nucleotides 130 to 2046 of SEQ ID NO: 3. In another aspect, the mature polypeptide coding sequence of the galactose oxidase is nucleotides 130 to 2040 of SEQ ID NO: 1, 5, or 7 or nucleotides 136 to 2046 of SEQ ID NO: 3 (e.g., when recombinantly expressed in A. oryzae as described in Xu, F. et al. Appl Biochem Biotechnol 2000, 88, 23-32).

Fragment: The term “fragment” means a polypeptide having one or more (e.g., two, several) amino acids deleted from the amino and/or carboxyl terminus of a referenced polypeptide sequence. In one aspect, the fragment has galactose oxidase activity. In another aspect, the number of amino acid residues in the fragment is at least 75%, e.g., at least 80%, 85%, 90%, or 95% of any galactose oxidase described herein, e.g., at least 75%, e.g., at least 80%, 85%, 90%, or 95% of the number of amino acid residues in the mature polypeptide sequence of SEQ ID NOs: 2, 4, 6, or 8.

Subsequence: The term “subsequence” means a polynucleotide having one or more (e.g., two, several) nucleotides deleted from the 5′ and/or 3′ end of the referenced nucleotide sequence. In one aspect, the subsequence encodes a fragment having galactose oxidase activity. In another aspect, the number of nucleotides residues in the subsequence is at least 75%, e.g., at least 80%, 85%, 90%, or 95% of the number of nucleotide residues in any sequence encoding a galactose oxidase described herein, e.g., at least 75%, e.g., at least 80%, 85%, 90%, or 95% of the number of nucleotide residues in the mature polypeptide coding sequence of SEQ ID NOs: 1, 3, 5, or 7.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Sequence Identity: The relatedness between two amino acid sequences or between two nucleotide sequences is described by the parameter “sequence identity”.

For purposes described herein, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes described herein, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Expression: The term “expression” includes any step involved in the production of the polypeptide including, but not limited to, transcription, post-transcriptional modification, translation, post-translational modification, and secretion. Expression can be measured—for example, to detect increased expression—by techniques known in the art, such as measuring levels of mRNA and/or translated polypeptide.

Nucleic acid construct: The term “nucleic acid construct” means a polynucleotide comprises one or more (e.g., two, several) control sequences. The polynucleotide may be single-stranded or double-stranded, and may be isolated from a naturally occurring gene, modified to contain segments of nucleic acids in a manner that would not otherwise exist in nature, or synthetic.

Control sequence: The term “control sequence” means a nucleic acid sequence necessary for polypeptide expression. Control sequences may be native or foreign to the polynucleotide encoding the polypeptide, and native or foreign to each other. Such control sequences include, but are not limited to, a leader sequence, polyadenylation sequence, propeptide sequence, promoter sequence, signal peptide sequence, and transcription terminator sequence. The control sequences may be provided with linkers for the purpose of introducing specific restriction sites facilitating ligation of the control sequences with the coding region of the polynucleotide encoding a polypeptide.

Operably linked: The term “operably linked” means a configuration in which a control sequence is placed at an appropriate position relative to the coding sequence of a polynucleotide such that the control sequence directs the expression of the coding sequence.

Expression vector: The term “expression vector” means a linear or circular DNA molecule that comprises a polynucleotide encoding a polypeptide and is operably linked to control sequences, wherein the control sequences provide for expression of the polynucleotide encoding the polypeptide. At a minimum, the expression vector comprises a promoter sequence, and transcriptional and translational stop signal sequences.

Host cell: The term “host cell” means any cell type that is susceptible to transformation, transfection, transduction, and the like with a nucleic acid construct or expression vector comprising one or more (e.g., two, several) polynucleotides described herein (e.g., a polynucleotide encoding a carbonic anhydrase). The term “host cell” encompasses any progeny of a parent cell that is not identical to the parent cell due to mutations that occur during replication.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

Mutant: The term “mutant” means a polynucleotide encoding a variant.

Parent or parent galactose oxidase: The term “parent” or “parent galactose oxidase” means a naturally occurring galactose oxidase which is used as a reference in producing the variants described herein.

Variant: The term “variant” means a polypeptide having galactose oxidase activity comprising an alteration, i.e., a substitution, insertion, and/or deletion, at one or more (e.g., two, several) positions compared to a parent. A substitution means replacement of the amino acid occupying a position with a different amino acid; a deletion means removal of the amino acid occupying a position; and an insertion means adding an amino acid adjacent to and immediately following the amino acid occupying a position. The variants described herein are not necessarily derived directly from the parent so long as the indicated alteration(s) with respect to the parent is present.

The variants have at least 20%, e.g., at least 40%, at least 50%, at least 60%, at least 70%, at least 80%, at least 90%, at least 95%, or at least 100% of the galactose oxidase activity of the mature polypeptide of SEQ ID NO: 2.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

### Conventions for Designation of Variants

For purposes of the galactose oxidase variants described herein, the mature polypeptide of SEQ ID NO: 2 is used to determine the corresponding amino acid residue in another galactose oxidase. The amino acid sequence of another galactose oxidase is aligned with the mature polypeptide of SEQ ID NO: 2, and based on the alignment, the amino acid position number corresponding to any amino acid residue in the mature polypeptide of SEQ ID NO: 2 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

For purposes of the peroxygenase variants described herein, the mature polypeptide of SEQ ID NO: 10 is used to determine the corresponding amino acid residue in another peroxygenase. The amino acid sequence of another peroxygenase is aligned with the mature polypeptide of SEQ ID NO: 10, and based on the alignment, the amino acid position number corresponding to any amino acid residue in the mature polypeptide of SEQ ID NO: 10 is determined using the Needleman-Wunsch algorithm as described supra.

Identification of the corresponding amino acid residue in another galactose oxidase or peroxygenase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other enzyme has diverged from the mature polypeptide of SEQ ID NO: 2 or SEQ ID NO: 10 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G-K-A |

Multiple Alterations.

Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different Alterations.

Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

### “Tyr167Gly+Arg170Gly”, “Tyr167Gly+Arg170Ala”, “Tyr167Ala+Arg170Gly”, and “Tyr167Ala+Arg170Ala”.

Reference to “about” a value or parameter herein includes aspects that are directed to that value or parameter per se. For example, description referring to “about X” includes the aspect “X”. When used in combination with measured values, “about” includes a range that encompasses at least the uncertainty associated with the method of measuring the particular value, and can include a range of plus or minus two standard deviations around the stated value.

As used herein and in the appended claims, the singular forms “a,” “or,” and “the” include plural referents unless the context clearly dictates otherwise. It is understood that the aspects described herein include “consisting” and/or “consisting essentially of” aspects.

Unless defined otherwise or clearly indicated by context, all technical and scientific terms used herein have the same meaning as commonly understood by one of ordinary skill in the art.

### DETAILED DESCRIPTION

Described herein, inter alia, are methods of oxidizing hydroxymethylfurfural (HMF) using galactose oxidase polypeptides and galactose oxidase variants.

### Galactose Oxidases and Polynucleotides Encoding Galactose Oxidases

The galactose oxidase used in the methods herein can be any galactose oxidase that is suitable for oxidizing HMF, such as a naturally occurring galactose oxidase or a variant thereof. As described in more detail below, the galactose oxidase may be recombinantly produced from any suitable host organism, e.g., Aspergillus oryzae or Fusarium venenatum (see Xu, F. et al. Appl Biochem Biotechnol 2000, 88, 23-32).

In some aspects, the galactose oxidase: (a) has at least 60% sequence identity (e.g., at least 70%, at least 75%, at least 80%, at least 85%, at least 90%, at least 95%, at least 97%, at least 98%, at least 99%, or 100% sequence identity) to the mature polypeptide sequence of SEQ ID NO: 2 or 4; (b) is encoded by a coding sequence that hybridizes under at least low, medium, medium-high, high, or very high stringency conditions with the full-length complementary strand of the mature polypeptide coding sequence of SEQ ID NO: 1 or 3; or (c) is encoded by a coding sequence that has at least 60% sequence identity (e.g., at least 70%, at least 75%, at least 80%, at least 85%, at least 90%, at least 95%, at least 97%, at least 98%, at least 99%, or 100% sequence identity) to the mature polypeptide coding sequence of SEQ ID NO: 1 or 3. In one aspect of the methods described herein, the galactose oxidase does not comprise the mature polypeptide sequence of SEQ ID NO: 2.

In one aspect, the galactose oxidase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to mature polypeptide sequence of SEQ ID NO: 2 or 4. In one aspect, the galactose oxidase sequence differs by no more than ten amino acids, e.g., by no more than five amino acids, by no more than four amino acids, by no more than three amino acids, by no more than two amino acids, or by one amino acid from the mature polypeptide sequence of SEQ ID NO: 2 or 4.

In one aspect, the galactose oxidase comprises or consists of the mature polypeptide sequence of SEQ ID NO: 2 or 4, an allelic variant thereof, or a fragment of the foregoing having galactose oxidase activity. In another aspect, the galactose oxidase comprises or consists of the mature polypeptide sequence of SEQ ID NO: 2 or 4. In another aspect, the galactose oxidase comprises or consists of amino acids 1 to 639 of SEQ ID NO: 2 or 4.

In one aspect, the galactose oxidase has an amino acid substitution, deletion, and/or insertion of one or more (e.g., two, several) amino acids of the mature polypeptide sequence of SEQ ID NO: 2 or 4. The amino acid changes are generally of a minor nature, that is conservative amino acid substitutions or insertions that do not significantly affect the folding and/or activity of the protein; small deletions, typically of one to about 30 amino acids; small amino-terminal or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

For galactose oxidase, the skilled artisan can use the teachings from the galactose oxidase crystal structure (Ito, N. et al. Nature 1991, 350, 87-90) and the teachings of the variant libraries known in the art (Lippow et al. Chem Biol 2010, 17, 1306-1315) together with the teachings of the present disclosure as guidance in identifying amino acid residues that may be altered without significantly changing activity.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the galactose oxidase, alter the substrate specificity, change the pH optimum, and the like. Examples of galactose oxidase variants with improved properties are described below.

Essential amino acids in a galactose oxidase can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for galactose oxidase activity to identify amino acid residues that are critical to the activity of the molecule. See also, Hilton et al., 1996, J. Biol. Chem. 271: 4699-4708. The active site of the galactose oxidase or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with other galactose oxidases that are related to the referenced galactose oxidase.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active galactose oxidases can be recovered from the host cells and rapidly sequenced using standard methods in the art. These methods allow the rapid determination of the importance of individual amino acid residues in a polypeptide.

In one aspect, the galactose oxidase is encoded by a coding sequence that hybridizes under at least low stringency conditions, e.g., medium stringency conditions, medium-high stringency conditions, high stringency conditions, or very high stringency conditions with the full-length complementary strand of the mature polypeptide coding sequence of SEQ ID NO: 1 or 3 (see, e.g., J. Sambrook, E. F. Fritsch, and T. Maniatus, 1989, Molecular Cloning, A Laboratory Manual, 2d edition, Cold Spring Harbor, N.Y.).

In one aspect, the galactose oxidase is encoded by a coding sequence that has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 1 or 3.

In one aspect, the galactose oxidase is encoded by a coding sequence that comprises or consists of the mature polypeptide coding sequence of SEQ ID NO: 1 or 3. In one aspect, the galactose oxidase is encoded by a coding sequence that comprises or consists of nucleotides 124 to 2040 of SEQ ID NO: 1 or nucleotides 130 to 2046 of SEQ ID NO: 3. In one aspect, the galactose oxidase is encoded by a coding sequence that comprises or consists of a subsequence of the mature polypeptide coding sequence of SEQ ID NO: 1 or 3, wherein the subsequence encodes a polypeptide having galactose oxidase activity. In one aspect, the number of nucleotides residues in the subsequence is at least 75%, e.g., at least 80%, 85%, 90%, or 95% of the number of nucleotide residues in the mature polypeptide coding sequence of SEQ ID NO: 1 or 3.

In one aspect, the galactose oxidase is a fragment of the mature polypeptide sequence of SEQ ID NO: 2 or 4, or a fragment of any aspect of SEQ ID NO: 2 or 4 described herein, wherein the fragment has galactose oxidase activity. In one aspect, the number of amino acid residues in the fragment is at least 75%, e.g., at least 80%, 85%, 90%, or 95% of the number of amino acid residues in the mature polypeptide sequence of SEQ ID NO: 2 or 4.

The galactose oxidase may be a fused polypeptide or cleavable fusion polypeptide in which another polypeptide is fused at the N-terminus or the C-terminus of the galactose oxidase. A fused polypeptide may be produced by fusing a polynucleotide encoding another polypeptide to a polynucleotide encoding the galactose oxidase. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Techniques used to isolate or clone a polynucleotide—such as a polynucleotide encoding a galactose oxidase—as well as any other polypeptide used in any of the aspects mentioned herein, are known in the art and include isolation from genomic DNA, preparation from cDNA, or a combination thereof. The cloning of the polynucleotides from such genomic DNA can be effected, e.g., by using the well known polymerase chain reaction (PCR) or antibody screening of expression libraries to detect cloned DNA fragments with shares structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used. The polynucleotides may be cloned from a strain such as Fusarium, or another or related organism, and thus, for example, may be an allelic or species variant of the polypeptide encoding region of the nucleotide sequence.

The polynucleotide of SEQ ID NO: 1 or 3, or a subsequence thereof; as well as the amino acid sequence of SEQ ID NO: 2 or 4; or a fragment thereof; may be used to design nucleic acid probes to identify and clone a galactose oxidase from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, e.g., at least 14 nucleotides, at least 25 nucleotides, at least 35 nucleotides, at least 70 nucleotides in lengths. The probes may be longer, e.g., at least 100 nucleotides, at least 200 nucleotides, at least 300 nucleotides, at least 400 nucleotides, at least 500 nucleotides in lengths. Even longer probes may be used, e.g., at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin).

A genomic DNA or cDNA library prepared from such other strains may be screened for DNA that hybridizes with the probes described above and encodes a polypeptide having galactose oxidase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO: 54, or a subsequence thereof, the carrier material may be used in a Southern blot.

For purposes of the probes described above, hybridization indicates that the polynucleotide hybridizes to a labeled nucleic acid probe corresponding to SEQ ID NO: 1 or 3, the full-length complementary strand thereof, or a subsequence of the foregoing; under very low to very high stringency conditions. Molecules to which the nucleic acid probe hybridizes under these conditions can be detected using, for example, X-ray film.

In one aspect, the nucleic acid probe is the mature polypeptide coding sequence of SEQ ID NO: 1 or 3, or a subsequence thereof. In another aspect, the nucleic acid probe is a polynucleotide that encodes the mature polypeptide sequence of SEQ ID NO: 2 or 4, or a fragment thereof.

### Galactose Oxidase Variants

In some aspects, the galactose oxidase comprises a substitution at one or more (e.g., two, several) positions corresponding to positions 326, 329, 330, and 406 of SEQ ID NO: 2. Additional galactose oxidase variants that can be used in the methods described herein include those described in Lippow et al. Chem Biol 2010, 17, 1306-1315, the content of which is hereby incorporated by reference with respect to the variant sequences therein.

The galactose oxidase variants may or may not retain galactose activity, so long as the variant is capable of oxidation of the indicated substrate (e.g., HMF) according to the referenced method.

In an embodiment, the variant has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent galactose oxidase.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the mature polypeptide sequence of SEQ ID NO: 2.

In another aspect, a variant comprises substitution at one or more (e.g., two, several) positions corresponding to positions 326, 329, 330, and 406 of SEQ ID NO: 2. In another aspect, a variant comprises a substitution at two positions corresponding to any of positions 326, 329, 330, and 406 of SEQ ID NO: 2. In another aspect, a variant comprises a substitution at three positions corresponding to any of positions 326, 329, 330, and 406 of SEQ ID NO: 2. In another aspect, a variant comprises a substitution at each position corresponding to positions 326, 329, 330, and 406 of SEQ ID NO: 2.

In another aspect, the variant comprises or consists of a substitution at a position corresponding to position 326. In another aspect, the amino acid at a position corresponding to position 326 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu. In another aspect, the variant comprises or consists of the substitution Q326E of the mature polypeptide of SEQ ID NO: 2.

In another aspect, the variant comprises or consists of a substitution at a position corresponding to position 329. In another aspect, the amino acid at a position corresponding to position 329 is substituted with Ala, Arg, Asn, Asp, Cys, Gln, Glu, Gly, His, Ile, Leu, Lys, Met, Phe, Pro, Ser, Thr, Trp, Tyr, or Val, preferably with Arg or Lys. In another aspect, the variant comprises or consists of the substitution Y329R/K of the mature polypeptide of SEQ ID NO: 2.

In another aspect, the variant comprises or consists of a substitution at a position corresponding to position 330. In another aspect, the amino acid at a position corresponding to position 330 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys. In another aspect, the variant comprises or consists of the substitution R330K of the mature polypeptide of SEQ ID NO: 2. In another aspect, the variant comprises or consists of a position corresponding to position 406. In another aspect, the amino acid at a position corresponding to position 406 is substituted with Ala, Arg, Asn, Asp, Cys, Gin, Glu, Gly, His, Ile, Leu, Lys, Met, Phe, Pro, Ser, Thr, Trp, Tyr, or Val, preferably with Thr, Arg, or Lys. In another aspect, the variant comprises or consists of the substitution Q406T/R/K of the mature polypeptide of SEQ ID NO: 2.

In another aspect, the variant comprises or consists of an alteration at positions corresponding to positions 326 and 329, such as those described above.

In another aspect, the variant comprises or consists of alterations at positions corresponding to positions 326 and 330, such as those described above.

In another aspect, the variant comprises or consists of alterations at positions corresponding to positions 326 and 406, such as those described above.

In another aspect, the variant comprises or consists of alterations at positions corresponding to positions 329 and 330, such as those described above.

In another aspect, the variant comprises or consists of alterations at positions corresponding to positions 329 and 406, such as those described above.

In another aspect, the variant comprises or consists of alterations at positions corresponding to positions 330 and 406, such as those described above.

In another aspect, the variant comprises or consists of alterations at positions corresponding to positions 326, 329, and 330, such as those described above.

In another aspect, the variant comprises or consists of alterations at positions corresponding to positions 326, 329, and 406, such as those described above.

In another aspect, the variant comprises or consists of alterations at positions corresponding to positions 326, 330, and 406, such as those described above.

In another aspect, the variant comprises or consists of alterations at positions corresponding to positions 329, 330, and 406, such as those described above.

In another aspect, the variant comprises or consists of alterations at positions corresponding to positions 326, 329, 330, and 406, such as those described above.

In another aspect, the variant comprises or consists of one or more (e.g., two, several) substitutions selected from Q326E, Y329K, R330K, and Q406T.

In another aspect, the variant comprises or consists of the substitutions Q326E+Y329R/K of the mature polypeptide of SEQ ID NO: 2.

In another aspect, the variant comprises or consists of the substitutions Q326E+R330K of the mature polypeptide of SEQ ID NO: 2.

In another aspect, the variant comprises or consists of the substitutions Q326E+Q406T/R/K of the mature polypeptide of SEQ ID NO: 2.

In another aspect, the variant comprises or consists of the substitutions Y329R/K+R330K of the mature polypeptide of SEQ ID NO: 2.

In another aspect, the variant comprises or consists of the substitutions Y329R/K+Q406T/R/K of the mature polypeptide of SEQ ID NO: 2.

In another aspect, the variant comprises or consists of the substitutions R330K+Q406T/R/K of the mature polypeptide of SEQ ID NO: 2.

In another aspect, the variant comprises or consists of the substitutions Q326E+Y329R/K+R330K of the mature polypeptide of SEQ ID NO: 2.

In another aspect, the variant comprises or consists of the substitutions Q326E+Y329R/K+Q406T/R/K of the mature polypeptide of SEQ ID NO: 2.

In another aspect, the variant comprises or consists of the substitutions Q326E+R330K+Q406T/R/K of the mature polypeptide of SEQ ID NO: 2.

In another aspect, the variant comprises or consists of the substitutions Y329R/K+R330K+Q406T/R/K of the mature polypeptide of SEQ ID NO: 2.

In another aspect, the variant comprises or consists of the substitutions Q326E+Y329R/K+R330K+Q406T/R/K of the mature polypeptide of SEQ ID NO: 2.

The variants may further comprise one or more additional substitutions at one or more (e.g., two, several) other positions, as described supra. For example, the variants may comprise one or more substitutions, such as substitutions corresponding to positions 290, 324, 333, 334, 383, 405, 441, and 463 of SEQ ID NO: 2 as described in Lippow et al. Chem Biol 2010, 17, 1306-1315.

In one embodiment, the variant has improved catalytic efficiency compared to the parent enzyme.

In another embodiment, the variant has improved catalytic rate compared to the parent enzyme.

In another embodiment, the variant has improved chemical stability compared to the parent enzyme.

In another embodiment, the variant has improved oxidation stability compared to the parent enzyme.

In another embodiment, the variant has improved pH activity compared to the parent enzyme.

In another embodiment, the variant has improved pH stability compared to the parent enzyme.

In another embodiment, the variant has improved specific activity compared to the parent enzyme.

In another embodiment, the variant has improved stability under storage conditions compared to the parent enzyme.

In another embodiment, the variant has improved substrate binding compared to the parent enzyme.

In another embodiment, the variant has improved substrate cleavage compared to the parent enzyme.

In another embodiment, the variant has improved substrate specificity compared to the parent enzyme.

In another embodiment, the variant has improved substrate stability compared to the parent enzyme.

In another embodiment, the variant has improved surface properties compared to the parent enzyme.

In another embodiment, the variant has improved thermal activity compared to the parent enzyme.

In another embodiment, the variant has improved thermostability compared to the parent enzyme.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used to prepare the variants, such as one of the many commercially available kits.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Peroxygenases

The peroxygenases used in the methods herein can be any peroxygenase that is suitable for oxidizing HMF, DFF, and/or FFCA, such as a naturally occurring peroxygenase or a variant thereof. As described in more detail below, the peroxygenase may be produced recombinantly produced from any suitable host organism, e.g., Aspergillus oryzae or Fusarium venenatum.

In some aspects, the peroxygenase has at least 60% sequence identity (e.g., at least 70%, at least 75%, at least 80%, at least 85%, at least 90%, at least 95%, at least 97%, at least 98%, at least 99%, or 100% sequence identity) to any one of SEQ ID NOs: 9, 10, 11, 12, 13, 14, 15, 16, 17, 18, 19, 20, 21, 22, 23, 24, 25, 26, 28, 29, 30, 31, or 32; or the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32.

In some aspects, the peroxygenase comprises an amino acid sequence represented by the motif: E-H-D-[G,A]-S-[L,I]-S-R (SEQ ID NO:27).

In one aspect, the peroxygenase sequence differs by no more than ten amino acids, e.g., by no more than five amino acids, by no more than four amino acids, by no more than three amino acids, by no more than two amino acids, or by one amino acid from the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32.

In one aspect, the peroxygenase comprises or consists of the mature polypeptide sequence of any one of SEQ ID NOs: 9, 10, 11, 12, 13, 14, 15, 16, 17, 18, 19, 20, 21, 22, 23, 24, 25, or 26, an allelic variant thereof, or a fragment of the foregoing having peroxygenase activity. In another aspect, the peroxygenase comprises or consists of any one of SEQ ID NOs: 9, 10, 11, 12, 13, 14, 15, 16, 17, 18, 19, 20, 21, 22, 23, 24, 25, 26, 28, 29, 30, 31, or 32.

In one aspect, the peroxygenase has an amino acid substitution, deletion, and/or insertion of one or more (e.g., two, several) amino acids of the mature polypeptide sequence of SEQ ID NO: 10, as described supra.

In one aspect, the peroxygenase is encoded by a coding sequence that hybridizes under at least low stringency conditions, e.g., medium stringency conditions, medium-high stringency conditions, high stringency conditions, or very high stringency conditions with the full-length complementary strand of the mature polypeptide coding sequence of SEQ ID NO: 9 (see, e.g., J. Sambrook, E. F. Fritsch, and T. Maniatus, 1989, supra).

In one aspect, the peroxygenase is a fragment of the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32, or a fragment of any related aspect described herein, wherein the fragment has peroxygenase activity. In one aspect, the number of amino acid residues in the fragment is at least 75%, e.g., at least 80%, 85%, 90%, or 95% of the number of amino acid residues in any one of SEQ ID NOs: 9, 10, 11, 12, 13, 14, 15, 16, 17, 18, 19, 20, 21, 22, 23, 24, 25, 26, 28, 29, 30, 31, or 32.

The peroxygenase may be a fused polypeptide or cleavable fusion polypeptide, as described supra.

Techniques used to isolate or clone a polynucleotide encoding a peroxygenase are described supra.

The amino acid sequence of any one of SEQ ID NOs: 9, 10, 11, 12, 13, 14, 15, 16, 17, 18, 19, 20, 21, 22, 23, 24, 25, 26, 28, 29, 30, 31, or 32; or a fragment thereof; may be used to design nucleic acid probes to identify and clone a peroxygenase from strains of different genera or species, as described supra.

Additional peroxygenases that can be used in the methods described herein include the peroxygenases described in WO2008/119780, the content of which is incorporated herein by reference.

### Peroxygenase Variants

In some aspects, the peroxygenase comprises a substitution at one or more (e.g., two, several) positions corresponding to positions 76, 134, and 201 of SEQ ID NO: 10. Peroxygenase variants of the Agrocybe aegeritae peroxygenase of SEQ ID NO: 9 and the Coprinopsis cinerea peroxygenase of SEQ ID NO: 10 have been described in U.S. Ser. No. 61/550,548, filed Oct. 24, 2011, the content of which is hereby incorporated by reference.

The peroxygenase variants may or may not retain peroxygenase activity, so long as the variant is capable of oxidation of the indicated substrate according to the referenced method.

In an embodiment, the variant has sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent peroxygenase.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the mature polypeptide sequence of SEQ ID NO: 10.

In another aspect, a variant comprises substitution at one or more (e.g., two, several) positions corresponding to positions 76, 134, and 201 of SEQ ID NO: 10. In another aspect, a variant comprises a substitution at two positions corresponding to any of positions 76, 134, or 201 of SEQ ID NO: 10. In another aspect, a variant comprises a substitution at each position corresponding to positions 76, 134, or 201 of SEQ ID NO: 10.

In another aspect, the variant comprises or consists of a substitution at a position corresponding to position 76. In another aspect, the amino acid at a position corresponding to position 326 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant comprises or consists of the substitution M76L of the mature polypeptide of SEQ ID NO: 10.

In another aspect, the variant comprises or consists of a substitution at a position corresponding to position 134. In another aspect, the amino acid at a position corresponding to position 134 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant comprises or consists of the substitution M134L of the mature polypeptide of SEQ ID NO: 10. In another aspect, the variant comprises or consists of the substitution M127L of the mature polypeptide of SEQ ID NO: 9.

In another aspect, the variant comprises or consists of a substitution at a position corresponding to position 201. In another aspect, the amino acid at a position corresponding to position 201 is substituted with Ala, Arg, Asn, Asp, Cys, Gln, Glu, Gly, His, Ile, Leu, Lys, Met, Phe, Pro, Ser, Thr, Trp, Tyr, or Val, preferably with Phe. In another aspect, the variant comprises or consists of the substitution Y201F of the mature polypeptide of SEQ ID NO: 10. In another aspect, the variant comprises or consists of the substitution Y194F of the mature polypeptide of SEQ ID NO: 9.

In another aspect, the variant comprises or consists of an alteration at positions corresponding to positions 76 and 134, such as those described above.

In another aspect, the variant comprises or consists of alterations at positions corresponding to positions 76 and 201, such as those described above.

In another aspect, the variant comprises or consists of alterations at positions corresponding to positions 134 and 201, such as those described above.

In another aspect, the variant comprises or consists of alterations at positions corresponding to positions 76, 134, and 201, such as those described above.

In another aspect, the variant comprises or consists of one or more (e.g., two, several) substitutions selected from M76L, M134L, and Y201F.

In another aspect, the variant comprises or consists of one or both substitutions selected from M127L and Y194F.

In another aspect, the variant comprises or consists of the substitutions M76L+M134L of the mature polypeptide of SEQ ID NO: 10.

In another aspect, the variant comprises or consists of the substitutions M76L+Y201F of the mature polypeptide of SEQ ID NO: 10.

In another aspect, the variant comprises or consists of the substitutions M134L+Y201F of the mature polypeptide of SEQ ID NO: 10.

In another aspect, the variant comprises or consists of the substitutions M76L+M134L+Y201F of the mature polypeptide of SEQ ID NO: 10.

In another aspect, the variant comprises or consists of the substitutions M127L+Y194F.

The variants may further comprise one or more additional substitutions at one or more (e.g., two, several) other positions, as described supra.

In one embodiment, the variant has improved catalytic efficiency compared to the parent enzyme.

In another embodiment, the variant has improved catalytic rate compared to the parent enzyme.

In another embodiment, the variant has improved chemical stability compared to the parent enzyme.

In another embodiment, the variant has improved oxidation stability compared to the parent enzyme.

In another embodiment, the variant has improved pH activity compared to the parent enzyme.

In another embodiment, the variant has improved pH stability compared to the parent enzyme.

In another embodiment, the variant has improved specific activity compared to the parent enzyme.

In another embodiment, the variant has improved stability under storage conditions compared to the parent enzyme.

In another embodiment, the variant has improved substrate binding compared to the parent enzyme.

In another embodiment, the variant has improved substrate cleavage compared to the parent enzyme.

In another embodiment, the variant has improved substrate specificity compared to the parent enzyme.

In another embodiment, the variant has improved substrate stability compared to the parent enzyme.

In another embodiment, the variant has improved surface properties compared to the parent enzyme.

In another embodiment, the variant has improved thermal activity compared to the parent enzyme.

In another embodiment, the variant has improved thermostability compared to the parent enzyme.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (e.g., several) mutations are introduced at one or more defined sites in a polynucleotide encoding the parent.

### Sources of Polypeptides

The galactose oxidases and peroxygenases described herein (e.g., a parent galactose oxidase) may be obtained from a microorganism of any genus. As used herein, the term “obtained from” in connection with a given source shall mean that the polypeptide encoded by a polynucleotide is produced by the source or by a cell in which the polynucleotide from the source has been inserted. In some aspects, the galactose oxidase or peroxygenase is produced by the source. In some aspects, the galactose oxidase or peroxygenase is not produced by the source and produced recombinantly by another species. As can be appreciated by one of skill in the art, the activity of a galactose oxidase or peroxygenase may be affected by the host cell in which it is produced, e.g., by post-translational modifications resulting from differences in cellular environment. In some aspects, the galactose oxidase or peroxygenase is expressed from a host other than any one of the sources described herein (e.g., the galactose oxidase may be expressed from a host other than Dactylium dendroides). In some aspects, the galactose oxidase or peroxygenase is produced from a heterologous polynucleotide, e.g., the galactose oxidase is expressed from a polynucleotide that is not native to the host cell.

The galactose oxidase or peroxygenase may be a bacterial galactose oxidase or peroxygenase. For example, the galactose oxidase or peroxygenase may be a Gram-positive bacterial galactose oxidase or peroxygenase such as a Bacillus, Streptococcus, Streptomyces, Staphylococcus, Enterococcus, Lactobacillus, Lactococcus, Clostridium, Geobacillus, or Oceanobacillus galactose oxidase or peroxygenase; or a Gram-negative bacterial galactose oxidase or peroxygenase such as an E. coli, Pseudomonas, Salmonella, Campylobacter, Helicobacter, Flavobacterium, Fusobacterium, Ilyobacter, Neisseria, or Ureaplasma galactose oxidase or peroxygenase.

In one aspect, the galactose oxidase or peroxygenase is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis galactose oxidase or peroxygenase.

In another aspect, the galactose oxidase or peroxygenase is a Streptococcus equisimilis, Streptococcus pyogenes, Streptococcus uberis, or Streptococcus equi subsp. Zooepidemicus galactose oxidase or peroxygenase. In another aspect, the galactose oxidase or peroxygenase is a Streptomyces achromogenes, Streptomyces avermitilis, Streptomyces coelicolor, Streptomyces griseus, or Streptomyces lividans galactose oxidase or peroxygenase.

The galactose oxidase or peroxygenase may be a fungal galactose oxidase or peroxygenase. In one aspect, the fungal galactose oxidase or peroxygenase is from a yeast such as a Candida, Kluyveromyces, Pichia, Saccharomyces, Schizosaccharomyces, or Yarrowia galactose oxidase, or a filamentous fungal galactose oxidase, such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria.

In another aspect, the galactose oxidase or peroxygenase is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis galactose oxidase or peroxygenase.

In another aspect, the galactose oxidase or peroxygenase is an Acremonium cellulolyticus, Aspergillus aculeatus, Aspergillus awamori, Aspergillus flavus, Aspergillus fumigatus, Aspergillus foetidus, Aspergillus japonicus, Aspergillus nidulans, Aspergillus niger, Aspergillus oryzae, Aspergillus sojae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium austroamericanum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, or Trichoderma viride galactose oxidase or peroxygenase.

In another aspect, the galactose oxidase is a Fusarium galactose oxidase, such as the Fusarium austroamericanum galactose oxidase of SEQ ID NO: 2.

In another aspect, the peroxygenase is a Agrocybe peroxygenase, such as the Agrocybe aegeritae peroxygenase of SEQ ID NO: 9.

In another aspect, the peroxygenase is a Coprinopsis peroxygenase, such as the Coprinopsis cinerea peroxygenase of SEQ ID NO: 10 or SEQ ID NO: 11.

In another aspect, the peroxygenase is an Aspergillus peroxygenase, such as the Aspergillus niger peroxygenase of SEQ ID NO: 12, SEQ ID NO: 13, SEQ ID NO: 14, or SEQ ID NO: 15; or the Aspergillus carbonarius peroxygenase of SEQ ID NO: 26.

In another aspect, the peroxygenase is a Poronia peroxygenase, such as the Poronia punctata peroxygenase of SEQ ID NO: 16.

In another aspect, the peroxygenase is a Chaetomium peroxygenase, such as the Chaetomium virescens peroxygenase of SEQ ID NO: 17, SEQ ID NO: 18, or SEQ ID NO: 28; or the Chaetomium globosum peroxygenase of SEQ ID NO: 21, SEQ ID NO: 22, SEQ ID NO: 23, or SEQ ID NO: 24.

In another aspect, the peroxygenase is a Humicola peroxygenase, such as the Humicola insolens peroxygenase of SEQ ID NO: 19 or SEQ ID NO: 20.

In another aspect, the peroxygenase is a Sclerotinia peroxygenase, such as the Sclerotinia sclerotiorum peroxygenase of SEQ ID NO: 25.

In another aspect, the peroxygenase is a Daldinia peroxygenase, such as the Daldinia caldariorum peroxygenase of SEQ ID NO: 29.

In another aspect, the peroxygenase is a Myceliophthora peroxygenase, such as the Myceliophthora fergusii peroxygenase of SEQ ID NO: 30; or the Myceliophthora hinnulea peroxygenase of SEQ ID NO: 31.

In another aspect, the peroxygenase is a Thielavia peroxygenase, such as the Thielavia hyrcaniae peroxygenase of SEQ ID NO: 32.

It will be understood that for the aforementioned species, both the perfect and imperfect states, and other taxonomic equivalents, e.g., anamorphs, are encompassed regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

The galactose oxidases and peroxygenases may also be identified and obtained from other sources including microorganisms isolated from nature (e.g., soil, composts, water, silage, etc.) or DNA samples obtained directly from natural materials (e.g., soil, composts, water, silage, etc.) using the above-mentioned probes. Techniques for isolating microorganisms and DNA directly from natural habitats are well known in the art. The polynucleotide encoding a galactose oxidase or peroxygenase may then be derived by similarly screening a genomic or cDNA library of another microorganism or mixed DNA sample. Once a polynucleotide encoding a galactose oxidase or peroxygenase has been detected with suitable probe(s) as described herein, the sequence may be isolated or cloned by utilizing techniques that are known to those of ordinary skill in the art (see, e.g., J. Sambrook, E. F. Fritsch, and T. Maniatus, 1989, Molecular Cloning, A Laboratory Manual, 2d edition, Cold Spring Harbor, N.Y.).

### Methods

In one aspect is a method of oxidizing 5-hydroxymethylfurfural (HMF), comprising contacting HMF with a galactose oxidase and/or a peroxygenase described herein in a reaction mixture under suitable conditions to provide an oxidized HMF product.

In one embodiment is a method of oxidizing 5-hydroxymethylfurfural (HMF), comprising contacting HMF with a galactose oxidase described herein in a reaction mixture under suitable conditions to provide 2,5-diformylfuran (DFF). The provided DFF may be the final intended product (e.g., DFF that is purified) or as an in situ intermediate to another intended product (e.g., as an intermediate oxidation state to a further oxidized product, such as formylfuran carboxylic acid (FFCA) or 2,5-furan dicarboxylic acid (FDCA)). In some embodiments, the reaction mixture further comprises a peroxygenase described herein, and DFF is further oxidized to formylfuran carboxylic acid (FFCA), 2,5-furan dicarboxylic acid (FDCA), a salt thereof, or a mixture of the foregoing.

Thus, in some embodiments are methods of oxidizing 5-hydroxymethylfurfural (HMF), comprising contacting HMF with a galactose oxidase described herein and a peroxygenase described herein in a reaction mixture under suitable conditions to provide formylfuran carboxylic acid (FFCA), 2,5-furan dicarboxylic acid (FDCA), a salt thereof, or a mixture of the foregoing. The provided FFCA and/or FDCA may be the final intended product(s) (e.g., FFCA and/or FDCA that is purified) or as in situ intermediates to another intended product.

In one embodiment is a method of oxidizing 5-hydroxymethylfurfural (HMF), comprising contacting HMF with a peroxygenase described herein in a reaction mixture under suitable conditions to provide 2,5-diformylfuran (DFF), 5-hydroxymethyl-2-furancarboxylic acid (HMFCA), formylfuran carboxylic acid (FFCA), 2,5-furan dicarboxylic acid (FDCA), a salt thereof, or a mixture of the foregoing. The provided DFF, HMFCA, FFCA and/or FDCA may be the final intended product(s) (e.g., purified) or as in situ intermediates to another intended product.

In another aspect is a method of oxidizing 2,5-diformylfuran (DFF), comprising contacting DFF with a peroxygenase described herein in a reaction mixture under suitable conditions to provide an oxidized DFF product. In one embodiment is a method of oxidizing 2,5-diformylfuran (DFF), comprising contacting DFF with a peroxygenase described herein in a reaction mixture under suitable conditions to provide formylfuran carboxylic acid (FFCA), 2,5-furan dicarboxylic acid (FDCA), a salt thereof, or a mixture of the foregoing. The provided FFCA and/or FDCA may be the final intended product(s) (e.g., FFCA and/or FDCA that is purified) or as in situ intermediates to another intended product.

In another aspect is a method of oxidizing 5-hydroxymethyl-2-furancarboxylic acid (HMFCA) or a salt thereof, comprising contacting HMFCA or a salt thereof with a galactose oxidase and/or a peroxygenase described herein in a reaction mixture under suitable conditions to provide an oxidized HMFCA product or a salt thereof.

In one embodiment is a method of oxidizing 5-hydroxymethyl-2-furancarboxylic acid (HMFCA) or a salt thereof, comprising contacting HMFCA or a salt thereof with a galactose oxidase described herein in a reaction mixture under suitable conditions to provide formylfuran carboxylic acid (FFCA) or a salt thereof. The provided FFCA may be the final intended product (e.g., FFCA that is purified) or as in situ intermediates to another intended product. In some of these embodiments, the reaction mixture further comprises a peroxygenase described herein. In some of these embodiments, the reaction mixture further comprises a peroxygenase described herein and FFCA is further oxidized to FDCA or a salt thereof.

In another embodiment is a method of oxidizing 5-hydroxymethyl-2-furancarboxylic acid (HMFCA) or a salt thereof, comprising contacting HMFCA or a salt thereof with a peroxygenase described herein in a reaction mixture under suitable conditions to provide formylfuran carboxylic acid (FFCA), 2,5-furan dicarboxylic acid (FDCA), a salt thereof, or a mixture of the foregoing. The provided FFCA and/or FDCA may be the final intended product(s) (e.g., FFCA and/or FDCA that is purified) or as in situ intermediates to another intended product.

In another aspect is a method of oxidizing formylfuran carboxylic acid (FFCA) or a salt thereof, comprising contacting FFCA or a salt thereof with a peroxygenase described herein in a reaction mixture under suitable conditions to provide 2,5-furan dicarboxylic acid (FDCA) or a salt thereof. The provided FDCA may be the final intended product(s) (e.g., FDCA that is purified) or as in situ intermediates to another intended product.

The reaction mixture can be any suitable reaction mixture for oxidation, such as a completely aqueous reaction mixture, or an aqueous reaction mixture comprising one or more organic solvents (e.g., organic solvents that are miscible with water to form a single phase system at standard conditions of 20° C. and 1 atm; or organic solvents that are not miscible with water). Suitable organic solvents, such as alcohols, nitriles, ethers, and ketones, can be determined by one skilled in the art.

In one aspect, the reaction mixture is primarily water, e.g., 50-100 v/v % of the aqueous liquid is water, 55-100 v/v % of the aqueous liquid is water, 60-100 v/v % of the aqueous liquid is water, 65-100 v/v % of the aqueous liquid is water, 70-100 v/v % of the aqueous liquid is water, 75-100 v/v % of the aqueous liquid is water, 80-100 v/v % of the aqueous liquid is water, 85-100 v/v % of the aqueous liquid is water, 90-100 v/v % of the aqueous liquid is water, or 95-100 v/v % of the aqueous liquid is water. Thus, in some aspects, the reaction mixture has less than 50 v/v % other organic solvents, e.g., in the range of 0-50 v/v %, 0-45 v/v %, 0-40 v/v %, 0-35 v/v %, 0-30 v/v %, 0-25 v/v %, 0-20 v/v %, 0-15 v/v %, 0-10 v/v %, or 0-5 v/v % organic solvent.

Suitable conditions used for the oxidation methods described herein may be determined by one skilled in the art in light of the teachings herein. In some aspects of the methods, the duration of the oxidation reaction is less than 48 hours, such as less than 36 hours, less than 24 hours, less than 12 hours, less than 8 hours, less than 6 hours, less than 4 hours, less than 2 hours, or less than 1 hour. The temperature is typically between about 10° C. to about 90° C., such as about 20° C. to about 60° C., about 20° C. to about 50° C., about 20° C. to about 40° C., or about room temperature, and at a pH of about 3.0 to about 10.0, such as about 3.0 to about 9.0, about 3.0 to about 7.0, about 3.0 to about 6.0, about 3.0 to about 5.0, about 3.5 to about 4.5, about 4.0 to about 8.0, about 4.0 to about 7.0, about 4.0 to about 6.0, about 4.0 to about 5.0, about 5.0 to about 8.0, about 5.0 to about 7.0, or about 5.0 to about 6.0, about 6.0 to about 8.0, about 6.0 to about 7.5, or about 6.0 to about 7.0, or about 6.5 to about 7.5, or about 5.0, about 5.5, about 6.0, about 6.5, about 7.0, about 7.5 or about 8.5. Suitable buffering agents are known in the art, such as carbonate, 1,4-piperazinediethanesulfonic acid (pIPES), 4-morpholinepropanesulfonic acid (MOPS), 4-(2-hydroxyethyl)-Ipiperazineethane-sulfonic acid (HEPES), triethanolamine, TRIS, phosphate and the like. In the context of the present invention the pH and temperature of the reaction mixture refers to any time in the oxidation process, such as t₀.

The methods using galactose oxidase may create by-products, such as hydrogen peroxide. The hydrogen peroxide byproduct may be eliminated or reduced, e.g., by use of a catalase or peroxidase to convert the hydrogen peroxide into water and oxygen, thereby minimizing unwanted oxidation of the enzyme and allowing increased yield. Exemplary catalases include Terminox, Terminox Ultra, Terminox Supreme, and Catazyme (Novozymes NS).

Any required oxygen used in the oxidation methods described herein may be supplied as oxygen from the atmosphere or an oxygen precursor for in situ production of oxygen. In many industrial applications, oxygen from the atmosphere will usually be present in sufficient quantity. If more O₂ is needed, supplemental oxygen may be added, e.g. as pressurized atmospheric air or as pure pressurized O₂. The catalase enzyme described supra may be used to generate oxygen from degradation of unwanted hydrogen peroxide.

The hydrogen peroxide required by the peroxygenase may be provided as an aqueous solution of hydrogen peroxide or a hydrogen peroxide precursor for in situ production of hydrogen peroxide. Any solid entity which liberates upon dissolution a peroxide, which is useable by peroxygenase, can serve as a source of hydrogen peroxide. Compounds which yield hydrogen peroxide upon dissolution in water or an appropriate aqueous based medium include but are not limited to metal peroxides, percarbonates, persulphates, perphosphates, peroxyacids, alkyperoxides, acylperoxides, peroxyesters, urea peroxide, perborates and peroxycarboxylic acids or salts thereof.

Another source of hydrogen peroxide is a hydrogen peroxide generating enzyme system, such as an oxidase (e.g., a galactose oxidase described herein) together with a substrate for the oxidase. Examples of combinations of oxidase and substrate comprise, but are not limited to, amino acid oxidase (see e.g., U.S. Pat. No. 6,248,575) and a suitable amino acid, glucose oxidase (see e.g., WO 95/29996) and glucose, lactate oxidase and lactate, galactose oxidase (see e.g., WO 00/50606) and galactose, and aldose oxidase (see e.g. WO 99/31990) and a suitable aldose.

By studying EC 1.1.3._, EC 1.2.3._, EC 1.4.3._, and EC 1.5.3._{—} or similar classes (under the International Union of Biochemistry), other examples of such combinations of oxidases and substrates are easily recognized by one skilled in the art.

Alternative oxidants which may be applied for peroxygenases may be oxygen combined with a suitable hydrogen donor like ascorbic acid, dehydroascorbic acid, dihydroxyfumaric acid or cysteine. An example of such oxygen hydrogen donor system is described by Pasta et al., Biotechnology & Bioengineering, (1999) vol. 62, issue 4, pp. 489-493.

Hydrogen peroxide or a source of hydrogen peroxide may be added at the beginning of or during the method of the invention, e.g. as one or more separate additions of hydrogen peroxide; or continuously as fed-batch addition. Typical amounts of hydrogen peroxide correspond to levels of from 0.001 mM to 25 mM, preferably to levels of from 0.005 mM to 5 mM, and particularly to levels of from 0.01 to 1 mM or 0.02 to 2 mM hydrogen peroxide. Hydrogen peroxide may also be used in an amount corresponding to levels of from 0.1 mM to 25 mM, preferably to levels of from 0.5 mM to 15 mM, more preferably to levels of from 1 mM to 10 mM, and most preferably to levels of from 2 mM to 8 mM hydrogen peroxide.

The reaction mixture may also contain one or more supplemental salts, such as an inorganic salt, to improve product yield and/or recovery. Exemplary salts include, but are not limited to metal halides, metal sulfates, metal sulfides, metal phosphates, metal nitrates, metal acetates, metal sulfites and metal carbonates, e.g., sodium chloride (NaCl), sodium sulfite (Na₂SO₃), magnesium chloride (MgCl₂), lithium chloride (LiCl), potassium chloride (KCl), calcium chloride (CaCl₂), cesium chloride (CsCl), sodium sulfate (Na₂SO₄), potassium sulfate (K₂SO₄), lithium bromide (LiBr), sodium bromide (NaBr), potassium bromide (KBr), lithium nitrate (LiNO₃), sodium nitrate (NaNO₃), potassium nitrate (KNO₃) and potassium iodine (KI).

In some aspects, the reaction mixture comprises copper, such as copper sulfate. In some of these aspects, the copper in the reaction mixture is at a concentration of less than or equal to 5 mM, such as less than or equal to 2.5 mM, less than or equal to 1 mM, less than or equal to 0.5 mM, less than or equal to 0.1 mM, less than or equal to 0.05 mM, less than or equal to 0.01 mM, less than or equal to 0.005 mM, less than or equal to 0.0015 mM, or less than or equal to 0.0005 mM.

The concentration of galactose oxidase for oxidation can be any suitable concentration, such as 0.005 mg/ml to 50 mg/ml, e.g., 0.01 mg/ml to 25 mg/ml, 0.05 mg/ml to 10 mg/ml, 0.1 mg/ml to 10 mg/ml, 0.1 mg/ml to 5 mg/ml, 0.005 mg/ml to 1 mg/ml, 0.01 mg/ml to 0.5 mg/ml, or 0.01 mg/ml to 0.05 mg/ml.

The concentration of peroxygenase for oxidation can be any suitable concentration, such as 0.005 mg/ml to 50 mg/ml, e.g., 0.01 mg/ml to 25 mg/ml, 0.05 mg/ml to 10 mg/ml, 0.1 mg/ml to 10 mg/ml, 0.1 mg/ml to 5 mg/ml, 0.005 mg/ml to 1 mg/ml, 0.01 mg/ml to 0.5 mg/ml, or 0.01 mg/ml to 0.05 mg/ml.

In some aspects of the methods described herein using galactose oxidase to oxidize HMF, at least 10%, e.g., at least 20%, at least 30%, at least 40%, at least 50%, at least 60%, at least 70%, at least 80%, at least 90%, at least 95%, at least 98%, at least 99%, or essentially all of the HMF is oxidized to DFF.

In some aspects of the methods described herein using galactose oxidase and peroxygenase to oxidize HMF, at least 10%, e.g., at least 20%, at least 30%, at least 40%, at least 50%, at least 60%, at least 70%, at least 80%, at least 90%, at least 95%, at least 98%, at least 99%, or essentially all of the HMF is oxidized to FFCA, FDCA, a salt thereof, or a mixture of the foregoing. In some of these aspects, at least 10%, e.g., at least 20%, at least 30%, at least 40%, at least 50%, at least 60%, at least 70%, at least 80%, at least 90%, at least 95%, at least 98%, at least 99%, or essentially all of the HMF is oxidized to FFCA or a salt thereof. In some of these aspects, at least 10%, e.g., at least 20%, at least 30%, at least 40%, at least 50%, at least 60%, at least 70%, at least 80%, at least 90%, at least 95%, at least 98%, at least 99%, or essentially all of the HMF is oxidized to FDCA or a salt thereof.

In some aspects of the methods described herein using galactose oxidase and/or peroxygenase to oxidize HMFCA or a salt thereof, at least 10%, e.g., at least 20%, at least 30%, at least 40%, at least 50%, at least 60%, at least 70%, at least 80%, at least 90%, at least 95%, at least 98%, at least 99%, or essentially all of the HMFCA a salt thereof is oxidized to FFCA, FDCA, a salt thereof, or a mixture of the foregoing. In some of these aspects, at least 10%, e.g., at least 20%, at least 30%, at least 40%, at least 50%, at least 60%, at least 70%, at least 80%, at least 90%, at least 95%, at least 98%, at least 99%, or essentially all of the HMFCA or salt thereof is oxidized to FFCA or a salt thereof. In some of these aspects, at least 10%, e.g., at least 20%, at least 30%, at least 40%, at least 50%, at least 60%, at least 70%, at least 80%, at least 90%, at least 95%, at least 98%, at least 99%, or essentially all of the HMFCA or salt thereof is oxidized to FDCA or a salt thereof.

In some aspects of the methods described herein using peroxygenase to oxidize HMF, at least 10%, e.g., at least 20%, at least 30%, at least 40%, at least 50%, at least 60%, at least 70%, at least 80%, at least 90%, at least 95%, at least 98%, at least 99%, or essentially all of the HMF is oxidized to FFCA, FDCA, a salt thereof, or a mixture of the foregoing. In some of these aspects, at least 10%, e.g., at least 20%, at least 30%, at least 40%, at least 50%, at least 60%, at least 70%, at least 80%, at least 90%, at least 95%, at least 98%, at least 99%, or essentially all of the HMF is oxidized to FFCA or a salt thereof.

In some of these aspects, at least 10%, e.g., at least 20%, at least 30%, at least 40%, at least 50%, at least 60%, at least 70%, at least 80%, at least 90%, at least 95%, at least 98%, at least 99%, or essentially all of the HMF is oxidized to FDCA or a salt thereof.

In some aspects of the methods described herein using peroxygenase to oxidize DFF, at least 10%, e.g., at least 20%, at least 30%, at least 40%, at least 50%, at least 60%, at least 70%, at least 80%, at least 90%, at least 95%, at least 98%, at least 99%, or essentially all of the DFF is oxidized to FFCA, FDCA, a salt thereof, or a mixture of the foregoing. In some of these aspects, at least 10%, e.g., at least 20%, at least 30%, at least 40%, at least 50%, at least 60%, at least 70%, at least 80%, at least 90%, at least 95%, at least 98%, at least 99%, or essentially all of the DFF is oxidized to FFCA or a salt thereof. In some of these aspects, at least 10%, e.g., at least 20%, at least 30%, at least 40%, at least 50%, at least 60%, at least 70%, at least 80%, at least 90%, at least 95%, at least 98%, at least 99%, or essentially all of the DFF is oxidized to FDCA or a salt thereof.

In some aspects of the methods described herein using peroxygenase to oxidize FFCA or a salt thereof, at least 10%, e.g., at least 20%, at least 30%, at least 40%, at least 50%, at least 60%, at least 70%, at least 80%, at least 90%, at least 95%, at least 98%, at least 99%, or essentially all of the FFCA or salt thereof is oxidized to FDCA, a salt thereof.

The starting material and/or product of the methods described herein may be in a non-salt form, or a salt, e.g., by the addition of a supplementary salt into the reaction mixture as described supra. The salt of a basic functional group of a compound may be prepared by methods known to those of skill in the art by treating the compound with an acid. The salt of an acidic functional group of a compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, bismuth salts, and calcium salts; ammonium salts; and aluminum salts. Examples of organic salts of acid compounds include, but are not limited to, procaine, dibenzylamine, N-ethylpiperidine, N,N′ dibenzylethylenediamine, trimethylamine, and triethylamine salts. Examples of inorganic salts of base compounds include, but are not limited to, hydrochloride and hydrobromide salts. Examples of organic salts of base compounds include, but are not limited to, tartrate, citrate, maleate, fumarate, and succinate.

The oxidized product of any of the methods described herein can be optionally recovered and purified from the reaction mixture using any procedure known in the art including, but not limited to, chromatography (e.g., size exclusion chromatography, adsorption chromatography, ion exchange chromatography), electrophoretic procedures, differential solubility, extraction (e.g., liquid-liquid extraction), pervaporation, extractive filtration, membrane filtration, membrane separation, reverse osmosis, ultrafiltration, or crystallization.

In some aspects of the methods, the oxidized product of any of the methods described herein before and/or after being optionally purified is substantially pure. With respect to the methods described herein, “substantially pure” intends a preparation of the referenced product (e.g., HMF, FFCA, or FDCA) that contains no more than 15% impurity, wherein impurity intends compounds other than the referenced product salt and non-salt forms. In one variation, a preparation of substantially pure DFF is provided wherein the preparation contains no more than 25% impurity, or no more than 20% impurity, or no more than 10% impurity, or no more than 5% impurity, or no more than 3% impurity, or no more than 1% impurity, or no more than 0.5% impurity.

Suitable assays to test for the production of the oxidized product described herein can be performed using methods known in the art. For example, the oxidized product (and other organic compounds) can be analyzed by methods such as Thin Layer Chromatography (TLC), HPLC (High Performance Liquid Chromatography), GC-MS (Gas Chromatography Mass Spectroscopy) and LC-MS (Liquid Chromatography-Mass Spectroscopy), NMR (Nuclear Magnetic Resonance) or other suitable analytical methods using routine procedures well known in the art.

The following examples are provided by way of illustration and are not intended to be limiting of the invention.

### EXAMPLES

Chemicals used as buffers and substrates were commercial products of at least reagent grade.

### Media

DAP4C-1 media was composed of 0.5 g yeast extract, 10 g maltose, 20 g dextrose, 11 g magnesium sulphate heptahydrate, 1 g dipotassium phosphate, 2 g citric acid monohydrate, 5.2 g potassium phosphate tribasic monohydrate, 1 ml Dowfax 63N10 (antifoaming agent), 2.5 g calcium carbonate, supplemented with 1 ml KU6 metal solution, and deionized water to 1000 ml.

KU6 metal solution was composed of 6.8 g ZnCl₂, 2.5 g CuSO₄.5H₂O (citric acid monohydrate), 0.13 g NiCl₂, 13.9 g FeSO₄.7H₂O, 8.45 g MnSO₄.H₂O, 3 g C₆H₈O₇.H₂O, and deionized water to 1000 ml.

PDA plates were composed of 39 g Potato Dextrose Agar and deionized water to 1000 ml.

LB plates were composed of 10 g of Bacto-Tryptone, 5 g of yeast extract, 10 g of sodium chloride, 15 g of Bacto-agar, and deionized water to 1000 ml.

LB medium was composed of 10 g of Bacto-Tryptone, 5 g of yeast extract, and 10 g of sodium chloride, and deionized water to 1000 ml.

COVE-Sucrose-T plates were composed of 342 g of sucrose, 20 g of agar powder, 20 ml of COVE salt solution, and deionized water to 1000 ml. The medium was sterilized by autoclaving at 15 psi for 15 minutes (Bacteriological Analytical Manual, 8th Edition, Revision A, 1998). The medium was cooled to 60° C. and 10 mM acetamide, Triton X-100 (50 μl/500 ml) was added.

COVE-N-Agar tubes were composed of 218 g Sorbitol, 10 g Dextrose, 2.02 g KNO₃, 25 g Agar, 50 ml Cove salt solution, and deionized water up to 1000 ml.

COVE salt solution was composed of 26 g of MgSO₄.7H₂O, 26 g of KCL, 26 g of KH₂PO₄, 50 ml of COVE trace metal solution, and deionized water to 1000 ml.

COVE trace metal solution was composed of 0.04 g of Na₂B₄O₇.10H₂O, 0.4 g of CuSO₄.5H₂O, 1.2 g of FeSO₄.7H₂O, 0.7 g of MnSO₄—H₂O, 0.8 g of Na₂MoO₄.2H₂O, 10 g of ZnSO₄.7H₂O, and deionized water to 1000 ml.

### Example 1

### Preparation of Galactose Oxidases and Peroxygenases

Fusarium austroamericanum (Dactylium dendroides, Fusarium graminearum) Galactose Oxidase

Non-recombinant (Dactylium dendroides): Galactose oxidase produced from the natural source Dactylium dendroides was purchased from Sigma-Aldrich (St. Louis, Mo., USA). Dactylium dendroides was reclassified as Fusarium graminearum, and then recognized as lineage 1 of the Fusarium graminearum complex, or Fusarium austroamericanum (see Cordeiro et al. J Basic Microbiol 2010, 50, 527-537).

Recombinant (Aspergillus oryzae): Recombinantly produced F. austroamericanum galactose oxidase expressed in an A. oryzae host was prepared by cloning and transformation of the coding sequence of SEQ ID NO: 1 (encoding the galactose oxidase of SEQ ID NO: 2) into A. oryzae as previously described (Xu, F. et al. Appl Biochem Biotechnol 2000, 88, 23-32).

Recombinant (Fusarium venenatum): Recombinantly produced F. austroamericanum galactose oxidase expressed in an F. venenatum host was prepared by cloning and transformation of the coding sequence of SEQ ID NO: 1 (encoding the galactose oxidase of SEQ ID NO: 2) into F. venenatum as previously described (Xu, F. et al. Appl Biochem Biotechnol 2000, 88, 23-32).

Fusarium austroamericanum Galactose Oxidase Variant “MutA”

The Fusarium austroamericanum galactose oxidase variant “MutA” differs from the wild-type enzyme at three positions with substitutions at Q326E, Y3289, and R330K; and is reported to have altered substrate specificity with relatively high activity on glucose (Lippow et al. Chem Biol 2010, 17, 1306-1315). To obtain the variant for testing and characterization, a synthetic gene coding for the variant was purchased, sub-cloned into and Aspergillus expression vector, and transformed into an Aspergillus oryzae expression strain.

The gene sequence of the wild-type enzyme was obtained from the public sequence record EMBL:M86819, trimmed to comprise the coding and Kozak sequences, and the codons for the substituted positions were modified to code for the substituted residues. HindIII and XhoI restriction sites were added at the 5′ and 3′ ends to facilitate subcloning, and the resulting edited DNA sequence (which comprises the coding sequence of SEQ ID NO: 5, which encodes the MutA variant of SEQ ID NO: 6) was ordered and purchased from GeneArt® (Life Technologies, Corp., Carlsbad, Calif., USA).

The synthetic gene coding for the MutA variant was subcloned into the Aspergillus expression vector pMStr57 (WO2004/032648) utilizing the HindIII and XhoI sites in the gene and vector, resulting in a MutA expression construct designated pMStr287. Vector pMStr57 contains sequences for selection and propagation in E. coli, and selection and expression in Aspergillus. Selection in Aspergillus is facilitated by the amdS gene of Aspergillus nidulans, which allows the use of acetamide as a sole nitrogen source. Expression in Aspergillus is mediated by a modified neutral amylase II (NA2) promoter from Aspergillus niger which is fused to the 5′ leader sequence of the triose phosphate isomerase (tpi) encoding-gene from Aspergillus nidulans, and the terminator from the amyloglucosidase-encoding gene from Aspergillus niger. The Aspergillus oryzae strain MT3568 (an amdS (acetamidase) disrupted derivative of JaL355 (WO 02/40694) in which pyrG auxotrophy was restored by disrupting the A. oryzae amdS gene with the pyrG gene) was transformed with construct pMStr287 using standard techniques, e.g. as described in WO2004/032648.

To identify transformants producing the galactose oxidase variant MutA, the transformants and MT3568 were cultured in 750 μl of three different media, YP+2% glucose (WO 05/066338), FG4P (WO 94/26925), and DAP4C-1, in 96-well deep-well microtiter plates with 1 ml well capacities. The cultures were incubated at 30° C. without shaking. Samples were taken after 4 days of growth and resolved with SDS-PAGE to monitor recombinant protein production. A single transformant was selected from among those tested for relatively high expression of the galactose oxidase variant as judged by comparing the intensity of the recombinant protein bands resolved in SDS-PAGE. The resulting transformant was isolated twice by dilution streaking conidia on selective medium containing 0.01% TRITON® X-100 to limit colony size.

Fusarium austroamericanum Galactose Oxidase Variant “MutB”

The Fusarium austroamericanum galactose oxidase variant “MutB” contains the three substitutions Q326E, Y3289, and R330K of MutA, and an additional substitution, Q406T, at a position identified by Lippow et al. (supra) as being involved in substrate specificity. To obtain the variant enzyme for testing and characterization, a synthetic gene coding for the variant was purchased, sub-cloned into and Aspergillus expression vector, and transformed into an Aspergillus oryzae expression strain.

The MutB peptide sequence was reverse translated with a method that preferentially utilizes codons that are frequently used in Aspergillus oryzae, and analyzes the resulting DNA sequences with algorithms designed to identify and remove sequence feature that might hinder cloning or expression. A single gene sequence was selected from this process, and the gene sequence file was completed by adding a translation-promoting Kozak sequence directly 5′ to the start codon, and BamHI and XhoI sites at the 5′ and 3′ ends to facilitate subcloning. The resulting DNA sequence (which comprises the coding sequence of SEQ ID NO: 7, which encodes the MutA variant of SEQ ID NO: 8) was ordered and purchased from GeneArt® (Life Technologies, Corp., Carlsbad, Calif., USA).

The synthetic gene coding for the MutB variant was subcloned into the Aspergillus expression vector pMStr57 (WO2004/032648) utilizing the BamHI and XhoI sites in the gene and vector, resulting in a MutB expression construct designated pMStr288. Selection in Aspergillus is facilitated by the amdS gene of Aspergillus nidulans, which allows the use of acetamide as a sole nitrogen source. Expression in Aspergillus is mediated by a modified neutral amylase II (NA2) promoter from Aspergillus niger which is fused to the 5′ leader sequence of the triose phosphate isomerase (tpi) encoding-gene from Aspergillus nidulans, and the terminator from the amyloglucosidase-encoding gene from Aspergillus niger. The Aspergillus oryzae strain MT3568 (supra) was transformed with pMStr288 using standard techniques, e.g. as described in WO2004/032648.

To identify transformants producing the galactose oxidase variant MutB, the transformants and MT3568 were cultured in 750 μl of three different media, YP+2% glucose (WO 05/066338), FG4P (WO 94/26925), and DAP4C-1, in 96-well deep-well microtiter plates with 1 ml well capacities. The cultures were incubated at 30° C. without shaking. Samples were taken after 4 days of growth and resolved with SDS-PAGE to monitor recombinant protein production. A single transformant was selected from among those tested for relatively high expression of the galactose oxidase variant as judged by comparing the intensity of the recombinant protein bands resolved in SDS-PAGE. The transformant was isolated twice by dilution streaking conidia on selective medium containing 0.01% TRITON® X-100 to limit colony size.

Fusarium longipes Galactose Oxidase

Fusarium longipes strain IM1179815 was used as the source of the galactose oxidase gene containing the coding sequence of SEQ ID NO: 3, which encodes the full-length Fusarium longipes galactose oxidase of SEQ ID NO: 4. Aspergillus oryzae MT3568 (supra) was used for heterologous expression of the gene encoding the Fusarium longipes galactose oxidase.

Cloning: The cloning primer set shown below (SEQ ID NO: 33 and 34) was designed to PCR-amplify the Fusarium longipes galactose oxidase coding sequence of SEQ ID NO: 3. A 5′ tag for InFusion cloning was added to the cloning primers according to the protocol described in the InFusion HD EcoDry Cloning Kit (Clontech Laboratories, Inc., Mountain View, Calif., USA) to fit cloning in the expression vector pDAu109 (WO 2005/042735).

### Primer 1:

(SEQ ID NO: 33)
5′-ACACA ACTGG GGATC CACCA TGAAA CAGCT CTTGA CACTT
GCTCT TTGCT TCAG-3′

### Primer 2:

(SEQ ID NO: 34)
5′-AGATC TCGAG AAGCT TATCG AGTAA CGCGA AGAGT CGTTG
CTACA CT-3′

The Fusarium longipes galactose oxidase gene coding sequence was amplified by PCR using the forward and reverse cloning primers described above with Fusarium longipes strain IM1179815 genomic DNA, previously prepared from mycelium grown on PDA plates with using a FastDNA Spin kit for soil (MP Biomedicals, Solon, Ohio, USA). The PCR was composed of 1 μl of genomic DNA, 2.5 μl of Primer 1 (10 μM), 2.5 μl of Primer 2 (10 μM), 10 μl of 5×HF buffer (Finnzymes Oy, Espoo, Finland), 1.6 μl of 50 mM MgCl₂, 2 μl of 10 mM dNTP, 0.5 μl of PHUSION® DNA polymerase (Finnzymes Oy, Espoo, Finland), and PCR-grade water to 50 μl. The amplification reaction was performed using a DYAD® Thermal Cycler (M.J. Research Inc. South San Francisco, Calif., USA) programmed for 2 minutes at 98° C. followed by 19 touchdown cycles each at 98° C. for 15 seconds, 70° C. (−1° C./cycle) for 30 seconds, and 72° C. for 2 minutes and 30 seconds; and 25 cycles each at 98° C. for 15 seconds, 60° C. for 30 seconds, 72° C. for 2 minutes and 30 seconds, and finally an extension of 5 minutes at 72° C.

The reaction products were isolated on 1.0% agarose gel electrophoresis using TAE buffer where an approximately 2.0 kb PCR band was excised from the gel and purified using a GFX® PCR DNA and Gel Band Purification Kit (GE Healthcare, HiHerod, Denmark) according to manufacturer's instructions. DNA corresponding to the Fusarium longipes galactose oxidase gene coding sequence was cloned into the expression vector pDAu109 (WO 2005/042735) linearized with Bam HI and Hind III, using an IN-FUSION™ Dry-Down PCR Cloning Kit (Clontech Laboratories, Inc., Mountain View, Calif., USA) according to the manufacturer's instructions.

A 2.5 μl volume of the diluted ligation mixture was used to transform E. coli TOP10 chemically competent cells (Invitrogen, Carlsbad, Calif., USA). Three colonies were selected on LB agar plates containing 100 μg of ampicillin per ml and cultivated overnight in 3 ml of LB medium supplemented with 100 μg of ampicillin per ml. Plasmid DNA was purified using a Qiagen Spin Miniprep kit (QIAGEN GmbH, Hilden, Germany) according to the manufacturer's instructions. The Fusarium longipes gene coding sequence was verified by Sanger sequencing before heterologous expression. The plasmid designated as IF395#2 (containing gene coding sequence of SEQ ID NO: 3) was selected for protoplast transformation and heterologous expression as described below.

Transformation: Protoplasts of Aspergillus oryzae MT3568 were prepared according to WO 95/002043. One hundred μl of protoplasts were mixed with 2.5-15 μg of the Aspergillus expression vector IF395#2 (supra) and 250 μl of 60% PEG 4000 (Applichem, Darmstadt, Germany) (polyethylene glycol, molecular weight 4,000), 10 mM CaCl₂, and 10 mM Tris-HCl pH 7.5 and gently mixed. The mixture was incubated at 37° C. for 30 minutes and the protoplasts were spread onto COVE plates for selection. After incubation for 4-7 days at 37° C. spores of eight transformants were inoculated into 0.5 ml of DAP-4C-1 medium (supplemented lactic acid and diammonium phosphate as described below) in 96 deep well plates. After 4 days cultivation at 30° C., the culture broths were analyzed by SDS-PAGE using Novex® 4-20% Tris-Glycine Gel (Invitrogen Corporation, Carlsbad, Calif., USA) to identify the transformants producing the largest amount of recombinant galactose oxidase from Fusarium longipes.

Spores of the best transformant were spread on COVE-Sucrose-T plates containing 0.01% TRITON® X-100 in order to isolate single colonies. The spreading was repeated twice in total on COVE-Sucrose-T plates, and then a single colony was spread on a COVE-N-Agar tube until sporulation.

Fermentation: 150 ml of DAP4C-1 media supplemented with 5 ml of 20% lactic acid, 3.5 ml of 50% diammonium phosphate, 1 ml copper (II) nitrate (150 mM) and spores from the best Aspergillus oryzae transformants above were cultivated in shake flasks during 4 days at a temperature of 30° C. under 100 rpm agitation. Culture broth was harvested by filtration using a 0.2 μm filter device.

Agrocybe Aeqeritae peoxmenase

Non-recombinant (Agrocybe Aegeritae; AaP): Peroxygenase corresponding to the mature polypeptide sequence of SEQ ID NO: 9 was produced from the natural source Agrocybe Aegeritae and isolated as previously described (Ullrich. et al. Appl Env Microbiol 2004, 70, 4575-4581).

Recombinant (Aspergillus oryzae; rAaP): Recombinantly produced A. Aegeritae peroxygenase corresponding to the mature polypeptide sequence of SEQ ID NO: 9 was prepared by expression in an A. oryzae host as described in WO 2008/119780.

Chaetomium virescens (Per21) Peroxygenase

Recombinantly produced C. virescens peroxygenase corresponding to the mature polypeptide sequence of SEQ ID NO: 28 was prepared as known in the art (e.g., see WO2013/021061, the content of which is hereby incorporated by reference).

Humicola insolens (Per27) Peroxygenase

Recombinantly produced H. insolens peroxygenase corresponding to the mature polypeptide sequence of SEQ ID NO: 19 was prepared as known in the art (e.g., see WO2013/021061, the content of which is hereby incorporated by reference).

Daldinia caldariorum (Per106) Peroxygenase

Recombinantly produced D. caldariorum peroxygenase corresponding to the mature polypeptide sequence of SEQ ID NO: 29 was prepared as known in the art (e.g., see WO2013/021061, the content of which is hereby incorporated by reference).

Myceliophthora fergusii (Per113) Peroxygenase

Recombinantly produced M. fergusii peroxygenase corresponding to the mature polypeptide sequence of SEQ ID NO: 30 was prepared as known in the art (e.g., see WO2013/021061, the content of which is hereby incorporated by reference).

Myceliophthora hinnulea (Per114) Peroxygenase

Recombinantly produced M. hinnulea peroxygenase corresponding to the mature polypeptide sequence of SEQ ID NO: 31 was prepared as known in the art (e.g., see WO2013/021061, the content of which is hereby incorporated by reference).

Thielavia hyrcaniae (Per117) Peroxygenase

Recombinantly produced T. hyrcaniae peroxygenase corresponding to the mature polypeptide sequence of SEQ ID NO: 32 was prepared as known in the art (e.g., see WO2013/021061, the content of which is hereby incorporated by reference).

### Example 2

### Screening of Oxidases for HMF Oxidation

Oxidations were carried out at 35° C. in open glass tubes for one hour in a 4 mL aqueous solution, comprising 1 mM HMF and the indicated amount of oxidase enzyme in 50 mM phosphate buffer (pH 7.5). The reaction mixture was stirred with a magnet in a thermostated heat block and oxygen was bubbled through the reaction mixture during the entire reaction. Samples were inactivated by heating to 75° C. for 5 minutes and centrifuged (13,000×g, 5 min.) prior to analysis.

Samples were analyzed on a GC/MS system consisting of a 7890A GC system equipped with a 5975C mass detector and a 7693 autosampler (Agilent, Santa Clara Calif., USA). Samples were injected in pulsed splitless mode on a DB-200 column (30 m, 250 μm, 0.25 μm) from Agilent J&W (Santa Clara Calif., USA) and eluted with 1.2 mL/min Helium using the following temperature program: 100° C. (for 1 min), 100-180° C. at 40° C./min, 180-220° C. at 20° C./min, 180-280° C. at 40° C./min, 280° C. (for 1 min.). The mass detector was operated in SIM mode monitoring ions 95, 97, 124 and 126 m/z. HMF and DFF were quantified by external calibration using authentic standards and calculated as the molar fraction of DFF (X_{DFF}=[DFF]/([DFF]+[HMF]) to account for any variation from solvent evaporation. Results are shown in Table 1.

TABLE 1

| Entry | Catalyst | [Enzyme] | X DFF |
|---|---|---|---|
| 1 | Blank (no enzyme) | — | 4% |
| 2 | Candida alcohol oxidase (A6941, sigma) | 10 mg ep/L | 4% |
| 3 | Pichia alcohol oxidase (A2404, Sigma) | 10 mg ep/L | 4% |
| 4 | Dactylium dendroides | 10 mg ep/L | 5% |
| | galactose oxidase (G7907, Sigma) | | |
| 5 | Athrobacter cholin oxidase (C4405, Sigma) | 10 mg ep/L | 5% |
| 6 | Fusarium austroamericanum | 10 mg ep/L | 29% |
| | galactose oxidase (recombinantly | | |
| | produced from Fusarium venenatum) | | |
| 7 | Fusarium austroamericanum | not known | 93% |
| | galactose oxidase variant mutB | | |
| 8 | 1 mM Cu(NO₃)₂ | — | 5% |

Under the conditions provided, the recombinantly produced F. austroamericanum galactose oxidase (the mature polypeptide of SEQ ID NO: 2) and the recombinantly produced F. austroamericanum galactose oxidase variant (the mature polypeptide of SEQ ID NO: 4) were each capable of significantly oxidizing HMF to DFF (29% and 93%, respectively). Interestingly, the non-recombinant version of this galactose oxidase (entry 4) was unable to significantly oxidize HMF beyond background levels.

### Example 3

### Activity of Dactylium dendroides Galactose Oxidase on HMF Oxidation

To further probe the lack of HMF oxidation by the galactose oxidase naturally expressed by Dactylium dendroides, additional reactions were conducted using desalted enzyme and enzyme supplemented with copper.

The Dactylium dendroides galactose oxidase from Sigma was dissolved in 10 mM phosphate buffer pH 6. A portion of the dissolved enzyme was desalted on a PD-10 desalting column (GE Healthcare Bio-Sciences Corp, Piscataway, N.J., USA) and an additional sample was supplemented with a stoichiometric amount of copper(II)sulfate. Oxidations were then carried out as described in Example 1, with samples taken at 30 min and 1 hour.

Samples were analyzed on an Agilent 1200 HPLC system equipped with a Diode Array Detector (Agilent, Santa Clara Calif., USA) and separated on a Synergi Fusion-RP (80 Å, 4 μm, 250×2 mm) column from Phenomenex (Torrance Calif., USA) thermostated at 60° C. Analytes were eluted with an isocratic eluent of aqueous 75 mM phosphoric acid containing 2% v/v of acetonitrile. HMF and DFF were quantified at 210 nm by external calibration using authentic standards and calculated as the molar fraction of DFF (X_{DFF}=[DFF]/([DFF]+[HMF]) to account for any variation from evaporation solvent evaporation. Results are shown in Table 2.

TABLE 2

| | | | X DFF | X DFF |
|---|---|---|---|---|
| Entry | Enzyme | [Enzyme] | 0.5 h | 1 h |
| 1 | Dactylium dendroides | 0.01 | 0% | 1% |
| | galactose oxidase (G7907, Sigma) | mg ep/mL | | |
| 2 | Dactylium dendroides | 0.01 | 0% | 1% |
| | galactose oxidase (G7907, Sigma) | mg ep/mL | | |
| | desalted | | | |
| 3 | Dactylium dendroides | 0.01 | 0% | 1% |
| | galactose oxidase (G7907, Sigma) | mg ep/mL | | |
| | desalted + 1.54 μM Cu | | | |
| 4 | blank (water) | — | 0% | 0% |

Under the conditions provided, the non-recombinant galactose oxidase produced by Dactylium dendroides was unable to significantly oxidize HMF beyond background levels despite enzyme desalting and supplemental copper in the reaction mixture.

### Example 4

### Screening of Galactose Oxidases for HMF Oxidation

Supernatants of recombinantly-produced galactose oxidase fermentations from Penicillium thomii, Penicillium chrysogenum, and Fusarium longipes were tested for oxidation activity on the HMF substrate.

Oxidations were carried out for 1 hour at 35° C. in open glass tubes using 4 mL aqueous solution of 1 mM HMF in 50 mM phosphate pH 6.5 buffer. Supernatants of the galactose oxidase fermentations were dosed at 50 μL per sample. The reaction mixture was stirred with a magnet in a thermostated heat block and oxygen was bubbled through the reaction mixture during the entire reaction. Samples were inactivated by heating to 75° C. for 5 minutes and centrifuged (13,000×g, 5 min.) prior to analysis. Galactose oxidases from Penicillium thomii and Penicillium chrysogenum both showed no activity on HMF compared to the control, whereas the Fusarium longipes galactose oxidase showed ˜1.1% molar fraction of DFF.

### Example 5

### Activity of Recombinant Fusarium austroamericanum Galactose Oxidase on HMF Oxidation at Various pH

Oxidations were carried out as in Example 2, using 0.005 mg ep/mL of the F. austroamericanum galactose oxidase variant of SEQ ID NO: 8 (mutB) in 50 mM phosphate buffer at the specified pH values. Samples were analyzed by HPLC as described in Example 3. Results are shown in Table 3.

TABLE 3

| pH | X DFF |
|---|---|
| 5.5 | 10% |
| 6.0 | 49% |
| 6.5 | 90% |
| 7.0 | 63% |
| 7.5 | 17% |
| 8.0 | 5% |

Under the conditions provided, the recombinantly produced F. austroamericanum galactose oxidase variant showed the highest oxidation of HMF at pH of about 6.5.

### Example 6

### Effect of Copper and Catalase to Activity of Galactose Oxidase on HMF Oxidation

Oxidations were carried out as in Example 2, with copper sulfate and/or Terminox® 200 L catalase (diluted 10,000 time in the sample) to the reaction mixture, as indicated in Table 4. Results indicated as “N.D.” were not determined.

TABLE 4

| | | Results: X DFF | | |
|---|---|---|---|---|
| | | 0.0015 mM | 0.5 mM | 1 mM |
| Entry | Enzyme | CuSO4 | CuSO4 | CuSO4 |
| 1 | F. austroamericanum | 16% | 6% | 5% |
| | galactose oxidase | | | |
| | (0.01 mg/mL) | | | |
| 2 | F. austroamericanum | 51% | 21% | 15% |
| | galactose oxidase | | | |
| | (0.055 mg/mL) | | | |
| 3 | F. austroamericanum | 56% | 25% | 24% |
| | galactose oxidase | | | |
| | (0.1 mg/mL) | | | |
| 4 | F. austroamericanum | 73% | N.D. | 24% |
| | galactose oxidase | | | |
| | (0.1 mg/mL) + catalase | | | |
| 5 | F. austroamericanum | 84% | 74% | 78% |
| | galactose oxidase | | | |
| | variant mutB (0.01 mg/mL) | | | |
| 6 | F. austroamericanum | 100% | 100% | 100% |
| | galactose oxidase | | | |
| | variant mutB (0.055 mg/mL) | | | |
| 7 | F. austroamericanum | 100% | 100% | 100% |
| | galactose oxidase | | | |
| | variant mutB (0.1 mg/mL) | | | |
| 8 | F. austroamericanum | 100% | N.D. | 100% |
| | galactose oxidase | | | |
| | variant mutB | | | |
| | (0.1 mg/mL) + catalase | | | |
| 9 | blank | <1% | <1% | <1% |

### Example 7

### DFF Oxidation by Peroxygenases at Various pH

Oxidations of 1 mM DFF were carried out with 1 mM H₂O₂ in 50 mM phosphate buffer at the specified pH using 0.01 mg ep/mL of one of the following peroxygenases: non-recombinant Agrocybe aegeritae (AaP), Agrocybe aegeritae recombinantly produced by Aspergillus oryzae (rAaP) or Humicola insolens (Per27). Reactions were performed at room temperature for 5 minutes and samples were then inactivated by heating at 75° C. for 5 minutes. Samples were analyzed by HPLC as in Example 3. Results are shown in Table 5 as the molar fraction of FFCA (X_{FFCA}=[FFCA]/([DFF]+[FFCA]).

TABLE 5

| pH | AaP | rAaP | Per27 |
|---|---|---|---|
| 5.5 | 26% | 11% | 3% |
| 6.0 | 27% | 16% | 4% |
| 6.5 | 35% | 19% | 4% |
| 7.0 | 33% | 22% | 5% |
| 7.5 | 25% | 16% | 4% |
| 8.0 | 14% | 9% | 4% |

### Example 8

### HMF Oxidation Using Combinations of Galactose Oxidase and Peroxygenase

Oxidations were carried out at 35° C. for 125 minutes in open glass tubes in a final volume of 4 mL aqueous solution, comprising 1 mM HMF and 0.005 mg/mL of the recombinantly produced F. austroamericanum galactose oxidase variant of SEQ ID NO: 8 (mutB) in 50 mM phosphate pH 6.5 buffer. Peroxygenase from Agrocybe aegeritae (AaP) was added as a single initial dose using 0.04 mg ep/mL or as a multi dose using an initial 0.04 mg ep/mL and adding additional 0.02 mg ep/mL doses after 25 and 60 minutes (for entries 4 and 6 only) or adding additional 0.04 mg ep/mL dose after 60 minutes (for entries 8 and 10 only). The reaction mixture was stirred with a magnet in a thermostated heat block and oxygen was bubbled through the reaction mixture during the entire reaction. After 5 minutes aqueous hydrogen peroxide (20 or 40 mM) was dosed in using a syringe pump (model 220-CE, World precision instruments, Aston, Stevenage, UK) until a total of 1, 1.5, 2 or 4 mM hydrogen peroxide had been reached. Samples were inactivated by heating to 75° C. for 5 minutes and centrifuged (13,000×g, 5 min.) and quantified by HPLC analysis as in 3 using external calibrations for HMF, DFF, FFCA, and FDCA. Results are shown in Table 6 as the molar fraction for each of the indicated products.

TABLE 6

| | | | | X | X | X | X |
|---|---|---|---|---|---|---|---|
| Entry | AaP | H₂O₂ | | HMF | DFF | FFCA | FDCA |
| 1 | none | none | | 17% | 83% | 0% | 0% |
| 2 | single dose | none | | 0% | 44% | 55% | 1% |
| 3 | single dose | 1 | mM | 0% | 0% | 85% | 15% |
| 4 | multi dose | 1 | mM | 0% | 0% | 67% | 33% |
| 5 | single dose | 1.5 | mM | 0% | 0% | 79% | 21% |
| 6 | multi dose | 1.5 | mM | 0% | 0% | 58% | 42% |
| 7 | single dose | 2 | mM | 0% | 1% | 76% | 23% |
| 8 | multi dose | 2 | mM | 0% | 0% | 55% | 45% |
| 9 | single dose | 4 | mM | 0% | 1% | 78% | 21% |
| 10 | multi dose | 4 | mM | 0% | 0% | 54% | 46% |

### Example 9

### HMF Oxidation by Peroxygenases

Oxidations of 1 mM HMF were carried out with 2 mM H₂O₂ in 10 mM phosphate buffer at pH 6.5 using 0.02 mg ep/mL of one of the following peroxygenases: Agrocybe aegeritae recombinantly produced by Aspergillus oryzae (rAaP), Chaetomium virescens (Per21), Humicola insolens (Per27), Daldinia caldariorum (Per106), Myceliophthora fergusii (Per113), Myceliophthora hinnulea (Per114) or Thielavia hyrcaniae (Per117).

Reactions were performed at room temperature for 120 minutes and then added catalase (Terminox Ultra 50L, Novozymes, Bagsvaerd, Denmark) to decompose residual H₂O₂. Samples were analyzed on an Agilent 1200 HPLC system equipped with a Diode Array Detector (Agilent, Santa Clara Calif., USA) and separated on a Synergi Fusion-RP (80 Å, 4 μm, 250×2 mm) column from Phenomenex (Torrance Calif., USA) thermostated at 60° C. Analytes were eluted with an isocratic eluent of aqueous 10 mM phosphate buffer pH 6.5 containing 2% v/v of acetonitrile. Analytes were quantified by external calibration using authentic standards at the following wavelengths: HMF (280 nm), DFF (280 nm), HMFCA (260 nm), FFCA (280 nm) and FDCA (260 nm). Results are shown in Table 7 as the molar fraction for each of the indicated products.

TABLE 7

| | | X | X | X | X | X |
|---|---|---|---|---|---|---|
| Entry | Enzyme | HMF | DFF | HMFCA | FFCA | FDCA |
| 1 | AaP | 84% | 4% | 10% | 1% | 0% |
| 2 | rAaP | 23% | 21% | 46% | 10% | 0% |
| 3 | Per 21 | 81% | 17% | 2% | 0% | 0% |
| 4 | Per 27 | 53% | 33% | 10% | 4% | 0% |
| 5 | Per 106 | 72% | 17% | 9% | 2% | 0% |
| 6 | per113 | 49% | 8% | 39% | 4% | 0% |
| 7 | Per 114 | 2% | 0% | 94% | 0% | 3% |
| 8 | Per117 | 51% | 38% | 5% | 5% | 0% |

### Example 10

### DFF Oxidation by Peroxygenases

Oxidations of 1 mM DFF were carried out with 2 mM H₂O₂ in 10 mM phosphate buffer at pH 6.5 using 0.02 mg ep/mL of one of the following peroxygenases: Agrocybe aegeritae recombinantly produced by Aspergillus oryzae (rAaP), Humicola insolens (Per27), Daldinia caldariorum (Per106), Myceliophthora fergusii (Per113), Myceliophthora hinnulea (Per114) or Thielavia hyrcaniae (Per117). Reactions were performed at room temperature for 120 minutes and then added catalase (Terminox Ultra 50L, Novozymes, Bagsvaerd, Denmark) to decompose residual H₂O₂. Samples were analyzed on an Agilent 1200 HPLC system equipped with a Diode Array Detector (Agilent, Santa Clara Calif., USA) and separated on a Synergi Fusion-RP (80 Å, 4 μm, 250×2 mm) column from Phenomenex (Torrance Calif., USA) thermostated at 60° C. Analytes were eluted with an isocratic eluent of aqueous 10 mM phosphate buffer pH 6.5 containing 2% v/v of acetonitrile. The following analytes were quantified by external calibration using authentic standards at the specified wavelengths: HMF (280 nm), DFF (280 nm), HMFCA (260 nm), FFCA (280 nm) and FDCA (260 nm). Results are shown in Table 8 as the molar fraction of each of the indicated products.

TABLE 8

| | | X | X | X | X | X |
|---|---|---|---|---|---|---|
| Entry | Enzyme | HMF | DFF | HMFCA | FFCA | FDCA |
| 1 | rAaP | 0% | 62% | 0% | 37% | 1% |
| 2 | Per 27 | 0% | 85% | 0% | 14% | 0% |
| 3 | Per 106 | 0% | 89% | 0% | 11% | 0% |
| 4 | per113 | 1% | 58% | 0% | 41% | 0% |
| 5 | Per 114 | 5% | 79% | 0% | 13% | 2% |
| 6 | Per117 | 0% | 85% | 0% | 15% | 0% |

### Example 11

### Additional Screening of Oxidases for HMF Oxidation

Oxidations were carried out as in example 2 (except using 50 mM phosphate buffer pH 6.5). Samples were analyzed on an Agilent 1200 HPLC system equipped with a Diode Array Detector (Agilent, Santa Clara Calif., USA) and separated on a Rezex ROA-Organic acid H+ (8 μm, 300×7.8 mm) column from Phenomenex (Torrance Calif., USA) thermostated at 70° C. Analytes were eluted with an isocratic eluent of aqueous 0.005N sulfuric acid. HMF and DFF were quantified at 280 nm by external calibration using authentic standards and calculated as the molar fraction of DFF (X_{DFF}=[DFF]/([DFF]+[HMF]) to account for any variation from evaporation solvent evaporation. Results are shown in Table 10.

TABLE 10

| Entry | Enzyme | [Enzyme] | X HMF | X DFF |
|---|---|---|---|---|
| 1 | Fusarium | ~10 mg | 81% | 19% |
| | austroamericanum galactose | ep/mL | | |
| | oxidase (recombinantly | | | |
| | produced from | | | |
| | Aspergillus oryzae) | | | |
| 2 | Fusarium | ~10 mg | 27% | 73% |
| | austroamericanum galactose | ep/mL | | |
| | oxidase variant mutB | | | |
| 3 | Fusarium | ~10 mg | 23% | 77% |
| | austroamericanum galactose | ep/mL | | |
| | oxidase variant mutA | | | |

Although the foregoing has been described in some detail by way of illustration and example for the purposes of clarity of understanding, it is apparent to those skilled in the art that any equivalent aspect or modification, may be practiced. Therefore, the description and examples should not be construed as limiting the scope of the invention.

The present invention may be further described by the following numbered paragraphs:
[1] A method of oxidizing 5-hydroxymethylfurfural (HMF), comprising contacting HMF with a galactose oxidase in a reaction mixture under suitable conditions to provide 2,5-diformylfuran (DFF).
[2] The method of paragraph [1], wherein the galactose oxidase: (a) has at least 60% sequence identity (e.g., at least 70%, at least 75%, at least 80%, at least 85%, at least 90%, at least 95%, at least 97%, at least 98%, at least 99%, or 100% sequence identity) to the mature polypeptide sequence of SEQ ID NO: 2; (b) is encoded by a coding sequence that hybridizes under at least low, medium, medium-high, high, or very high stringency conditions with the full-length complementary strand of the mature polypeptide coding sequence of SEQ ID NO: 1; or (c) is encoded by a coding sequence that has at least 60% sequence identity (e.g., at least 70%, at least 75%, at least 80%, at least 85%, at least 90%, at least 95%, at least 97%, at least 98%, at least 99%, or 100% sequence identity) to the mature polypeptide coding sequence of SEQ ID NO: 1.
[3] The method of paragraph [1], wherein the galactose oxidase has at least 60% sequence identity (e.g., at least 70%, at least 75%, at least 80%, at least 85%, at least 90%, at least 95%, at least 97%, at least 98%, at least 99%, or 100% sequence identity) to the mature polypeptide sequence of SEQ ID NO: 2.
[4] The method of paragraph [1], wherein the galactose oxidase comprises or consists of the mature polypeptide sequence of SEQ ID NO: 2. [5] The method of any one of paragraphs [1]-[4], wherein the mature polypeptide sequence is amino acids 1 to 639 of SEQ ID NO: 2.
[6] The method of paragraph [1], wherein the galactose oxidase is encoded by a coding sequence that hybridizes under at least low, medium, medium-high, high, or very high stringency conditions with the full-length complementary strand of the mature polypeptide coding sequence of SEQ ID NO: 1.
[7] The method of paragraph [1], wherein the galactose oxidase is encoded by a coding sequence that has at least 60% sequence identity (e.g., at least 70%, at least 75%, at least 80%, at least 85%, at least 90%, at least 95%, at least 97%, at least 98%, at least 99%, or 100% sequence identity) to the mature polypeptide coding sequence of SEQ ID NO: 1.
[8] The method of paragraph [1], wherein the galactose oxidase is encoded by a coding sequence that comprises or consists of the mature polypeptide coding sequence of SEQ ID NO: 1.
[9] The method of any one of paragraphs [1]-[8], wherein the galactose oxidase is a variant of a parent galactose oxidase comprising a substitution at one or more (several) positions corresponding to positions 326, 329, 330, and 406 of SEQ ID NO: 2.
[10] The method of any one of paragraphs [1]-[8], wherein the galactose oxidase is a variant of a parent galactose oxidase, comprising a substitution at a position corresponding to position 326 of SEQ ID NO: 2.
[11] The method of paragraph [10], wherein the substitution at a position corresponding to position 326 of SEQ ID NO: 2 is with Glu.
[12] The method of paragraph [10], wherein the substitution at a position corresponding to position 326 of SEQ ID NO: 2 is Q326E.
[13] The method of any one of paragraphs [1]-[12], wherein the galactose oxidase is a variant of a parent galactose oxidase, comprising a substitution at a position corresponding to position 329 of SEQ ID NO: 2.
[14] The method of paragraph [13], wherein the substitution at a position corresponding to position 329 of SEQ ID NO: 2 is with Arg or Lys. [15] The method of paragraph [13], wherein the substitution at a position corresponding to position 329 of SEQ ID NO: 2 is Y329R/K.
[16] The method of any one of paragraphs [1]-[15], wherein the galactose oxidase is a variant of a parent galactose oxidase, comprising a substitution at a position corresponding to position 330 of SEQ ID NO: 2.
[17] The method of paragraph [16], wherein the substitution at a position corresponding to position 330 of SEQ ID NO: 2 is with Lys.
[18] The method of paragraph [16], wherein the substitution at a position corresponding to position 330 of SEQ ID NO: 2 is R330K.
[19] The method of any one of paragraphs [1]-[18], wherein the galactose oxidase is a variant of a parent galactose oxidase, comprising a substitution at a position corresponding to position 406 of SEQ ID NO: 2.
[20] The method of paragraph [19], wherein the substitution at a position corresponding to position 406 of SEQ ID NO: 2 is with Thr, Arg, or Lys.
[21] The method of paragraph [19], wherein the substitution at a position corresponding to position 406 of SEQ ID NO: 2 is Q406T/R/K.
[22] The method of any one of paragraphs [1]-[21], wherein the galactose oxidase is a variant of a parent galactose oxidase, comprising a substitution at any two positions corresponding to positions 326, 329, 330, or 406 of SEQ ID NO: 2.
[23] The method of any one of paragraphs [1]-[21], wherein the galactose oxidase is a variant of a parent galactose oxidase, comprising a substitution at any three positions corresponding to positions 326, 329, 330, or 406 of SEQ ID NO: 2.
[24] The method of any one of paragraphs [1]-[21], wherein the galactose oxidase is a variant of a parent galactose oxidase, comprising a substitution at each position corresponding to positions 326, 329, and 330 of SEQ ID NO: 2.
[25] The method of any one of paragraphs [1]-[21], wherein the galactose oxidase is a variant of a parent galactose oxidase, comprising a substitution at each position corresponding to positions 326, 329, 330, and 406 of SEQ ID NO: 2. [26] The method of any one of paragraphs [9]-[25], wherein the variant galactose oxidase has improved catalytic efficiency or catalytic rate relative to the parent galactose oxidase.
[27] The method of any one of paragraphs [9]-[26], wherein the galactose oxidase variant comprises or consists of the mature polypeptide sequence of SEQ ID NO: 6.
[28] The method of paragraph [27], wherein the mature polypeptide sequence is amino acids 1 to 639 of SEQ ID NO: 6.
[29] The method of any one of paragraphs [9]-[26], wherein the galactose oxidase variant comprises or consists of the mature polypeptide sequence of SEQ ID NO: 8.
[30] The method of paragraph [29], wherein the mature polypeptide sequence is amino acids 1 to 639 of SEQ ID NO: 8.
[31] The method of any one of paragraphs [1]-[30], wherein the galactose oxidase is expressed from a heterologous polynucleotide.
[32] The method of any one of paragraphs [1]-[31], wherein the galactose oxidase is expressed from a host other than Fusarium austroamericanum.
[33] The method of paragraph [32], wherein the galactose oxidase is expressed from an Aspergillus oryzae host.
[34] The method of paragraph [32], wherein the galactose oxidase is expressed from a Fusarium venenatum host.
[35] The method of any one of paragraphs [1]-[34], wherein the galactose oxidase does not comprise the mature polypeptide sequence of SEQ ID NO: 2.
[36] The method of any one of paragraphs [1]-[35], wherein the reaction mixture further comprises a catalase.
[37] The method of any one of paragraphs [1]-[36], wherein the reaction mixture further comprises copper.
[38] The method of any one of paragraphs [1]-[36], wherein the reaction mixture further comprises copper sulfate.
[39] The method of paragraph [37] or [38], wherein the copper is at a concentration of less than or equal to 1 mM, e.g., less than or equal to 0.5 mM, or less than or equal to 0.0015 mM.
[40] The method of any one of paragraphs [1]-[39], wherein at least 10%, e.g., at least 20%, at least 30%, at least 40%, at least 50%, at least 60%, at least 70%, at least 80%, at least 90%, or at least 95% of the HMF is converted to DFF.
[41] The method of any one of paragraphs [1]-[40], wherein the reaction mixture further comprises a peroxygenase, and DFF is further oxidized to formylfuran carboxylic acid (FFCA), 2,5-furan dicarboxylic acid (FDCA), a salt thereof, or a mixture of the foregoing.
[42] The method of paragraph [41], wherein the peroxygenase has at least 60% sequence identity (e.g., at least 70%, at least 75%, at least 80%, at least 85%, at least 90%, at least 95%, at least 97%, at least 98%, at least 99%, or 100% sequence identity) to the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32.
[43] The method of paragraph [42], wherein the mature polypeptide sequence comprises the motif: E-H-D-[G,A]-S-[L,I]-S-R (SEQ ID NO:27).
[44] The method of paragraph [42] or [43], wherein the peroxygenase has at least 60% sequence identity (e.g., at least 70%, at least 75%, at least 80%, at least 85%, at least 90%, at least 95%, at least 97%, at least 98%, at least 99%, or 100% sequence identity) to the mature polypeptide sequence of SEQ ID NO: 9.
[45] The method of paragraph [44], wherein the peroxygenase comprises or consists of the mature polypeptide sequence of SEQ ID NO: 9.
[46] The method of paragraph [45], wherein the mature polypeptide sequence is amino acids 1 to 328 of SEQ ID NO: 9.
[47] The method of any one of paragraphs [41]-[44], wherein the peroxygenase is a variant of a parent peroxygenase comprising a substitution at one or more (several) positions corresponding to positions 76, 134, and 201 of SEQ ID NO: 10.
[48] The method of any one of paragraphs [41]-[44], wherein the peroxygenase is a variant of a parent peroxygenase, comprising a substitution at a position corresponding to position 76 of SEQ ID NO: 10.
[49] The method of paragraph [48], wherein the substitution at a position corresponding to position 76 of SEQ ID NO: 10 is with Leu.
[50] The method of paragraph [48], wherein the substitution at a position corresponding to position 76 of SEQ ID NO: 10 is M76L.
[51] The method of any one of paragraphs [41]-[44], wherein the peroxygenase is a variant of a peroxygenase, comprising a substitution at a position corresponding to position 134 of SEQ ID NO: 10.
[52] The method of paragraph [51], wherein the substitution at a position corresponding to position 134 of SEQ ID NO: 10 is with Leu.
[53] The method of paragraph [51], wherein the substitution at a position corresponding to position 134 of SEQ ID NO: 10 is M134L or M127L.
[54] The method of any one of paragraphs [41]-[44], wherein the peroxygenase is a variant of a parent peroxygenase, comprising a substitution at a position corresponding to position 201 of SEQ ID NO: 10.
[55] The method of paragraph [54], wherein the substitution at a position corresponding to position 201 of SEQ ID NO: 10 is with Phe.
[56] The method of paragraph [54], wherein the substitution at a position corresponding to position 201 of SEQ ID NO: 10 is Y201F or Y194F.
[57] The method of any one of paragraphs [47]-[56], wherein the peroxygenase is a variant of a parent peroxygenase, comprising a substitution at any two positions corresponding to positions 76, 134, and 201 of SEQ ID NO: 10.
[58] The method of any one of paragraphs [47]-[56], wherein the peroxygenase is a variant of a parent peroxygenase, comprising a substitution at each position corresponding to positions 76, 134, and 201 of SEQ ID NO: 10.
[59] The method of any one of paragraphs [41]-[58], wherein at least 10%, e.g., at least 20%, at least 30%, at least 40%, at least 50%, at least 60%, at least 70%, at least 80%, at least 90%, at least 95%, at least 97%, at least 98%, or at least 99% of the HMF is converted to formylfuran carboxylic acid (FFCA), 2,5-furan dicarboxylic acid (FDCA), a salt thereof, or a mixture of the foregoing.
[60] The method of any one of paragraphs [41]-[59], wherein the reaction mixture further comprises supplemental H₂O₂.
[61] A method of oxidizing 5-hydroxymethyl-2-furancarboxylic acid (HMFCA) or a salt thereof, comprising contacting HMFCA or a salt thereof with a galactose oxidase in a reaction mixture under suitable conditions to provide formylfuran carboxylic acid (FFCA) or a salt thereof.
[62] The method of paragraph [61], wherein the galactose oxidase: (a) has at least 60% sequence identity (e.g., at least 70%, at least 75%, at least 80%, at least 85%, at least 90%, at least 95%, at least 97%, at least 98%, at least 99%, or 100% sequence identity) to the mature polypeptide sequence of SEQ ID NO: 2; (b) is encoded by a coding sequence that hybridizes under at least low, medium, medium-high, high, or very high stringency conditions with the full-length complementary strand of the mature polypeptide coding sequence of SEQ ID NO: 1; or (c) is encoded by a coding sequence that has at least 60% sequence identity (e.g., at least 70%, at least 75%, at least 80%, at least 85%, at least 90%, at least 95%, at least 97%, at least 98%, at least 99%, or 100% sequence identity) to the mature polypeptide coding sequence of SEQ ID NO: 1.
[63] The method of paragraph [61], wherein the galactose oxidase has at least 60% sequence identity (e.g., at least 70%, at least 75%, at least 80%, at least 85%, at least 90%, at least 95%, at least 97%, at least 98%, at least 99%, or 100% sequence identity) to the mature polypeptide coding sequence of SEQ ID NO: 2.
[64] The method of paragraph [61], wherein the galactose oxidase comprises or consists of the mature polypeptide coding sequence of SEQ ID NO: 2.
[65] The method of any one of paragraphs [61]-[64], wherein the mature polypeptide sequence is amino acids 1 to 639 of SEQ ID NO: 2.
[66] The method of paragraph [61], wherein the galactose oxidase is encoded by a coding sequence that hybridizes under at least low, medium, medium-high, high, or very high stringency conditions with the full-length complementary strand of the mature polypeptide coding sequence of SEQ ID NO: 1.
[67] The method of paragraph [61], wherein the galactose oxidase is encoded by a coding sequence that has at least 60% sequence identity (e.g., at least 70%, at least 75%, at least 80%, at least 85%, at least 90%, at least 95%, at least 97%, at least 98%, at least 99%, or 100% sequence identity) to the mature polypeptide coding sequence of SEQ ID NO: 1.
[68] The method of paragraph [61], wherein the galactose oxidase is encoded by a coding sequence that comprises or consists of the mature polypeptide coding sequence of SEQ ID NO: 1.
[69] The method of any one of paragraphs [61]-[67], wherein the galactose oxidase is a variant of a parent galactose oxidase comprising a substitution at one or more (several) positions corresponding to positions 326, 329, 330, and 406 of SEQ ID NO: 2.
[70] The method of any one of paragraphs [61]-[67], wherein the galactose oxidase is a variant of a parent galactose oxidase, comprising a substitution at a position corresponding to position 326 of SEQ ID NO: 2.
[71] The method of paragraph [70], wherein the substitution at a position corresponding to position 326 of SEQ ID NO: 2 is with Glu.
[72] The method of paragraph [70], wherein the substitution at a position corresponding to position 326 of SEQ ID NO: 2 is Q326E.
[73] The method of any one of paragraphs [61]-[72], wherein the galactose oxidase is a variant of a parent galactose oxidase, comprising a substitution at a position corresponding to position 329 of SEQ ID NO: 2.
[74] The method of paragraph [73], wherein the substitution at a position corresponding to position 329 of SEQ ID NO: 2 is with Arg or Lys.
[75] The method of paragraph [73], wherein the substitution at a position corresponding to position 329 of SEQ ID NO: 2 is Y329R/K.
[76] The method of any one of paragraphs [61]-[75], wherein the galactose oxidase is a variant of a parent galactose oxidase, comprising a substitution at a position corresponding to position 330 of SEQ ID NO: 2. [77] The method of paragraph [76], wherein the substitution at a position corresponding to position 330 of SEQ ID NO: 2 is with Lys.
[78] The method of paragraph [76], wherein the substitution at a position corresponding to position 330 of SEQ ID NO: 2 is R330K.
[79] The method of any one of paragraphs [61]-[78], wherein the galactose oxidase is a variant of a parent galactose oxidase, comprising a substitution at a position corresponding to position 406 of SEQ ID NO: 2.
[80] The method of paragraph [79], wherein the substitution at a position corresponding to position 406 of SEQ ID NO: 2 is with Thr, Arg, or Lys.
[81] The method of paragraph [79], wherein the substitution at a position corresponding to position 406 of SEQ ID NO: 2 is Q406T/R/K.
[82] The method of any one of paragraphs [61]-[81], wherein the galactose oxidase is a variant of a parent galactose oxidase, comprising a substitution at any two positions corresponding to positions 326, 329, 330, or 406 of SEQ ID NO: 2.
[83] The method of any one of paragraphs [61]-[82], wherein the galactose oxidase is a variant of a parent galactose oxidase, comprising a substitution at any three positions corresponding to positions 326, 329, 330, or 406 of SEQ ID NO: 2.
[84] The method of any one of paragraphs [61]-[83], wherein the galactose oxidase is a variant of a parent galactose oxidase, comprising a substitution at each position corresponding to positions 326, 329, and 330 of SEQ ID NO: 2.
[85] The method of any one of paragraphs [61]-[84], wherein the galactose oxidase is a variant of a parent galactose oxidase, comprising a substitution at each position corresponding to positions 326, 329, 330, and 406 of SEQ ID NO: 2.
[86] The method of any one of paragraphs [61]-[85], wherein the variant galactose oxidase has improved catalytic efficiency or catalytic rate relative to the parent galactose oxidase.
[87] The method of any one of paragraphs [61]-[86], wherein the galactose oxidase variant comprises or consists of the mature polypeptide sequence of SEQ ID NO: 6.
[88] The method of paragraph [87], wherein the mature polypeptide sequence is amino acids 1 to 639 of SEQ ID NO: 6.
[89] The method of any one of paragraphs [61]-[86], wherein the galactose oxidase variant comprises or consists of the mature polypeptide sequence of SEQ ID NO: 8.
[90] The method of paragraph [89], wherein the mature polypeptide sequence is amino acids 1 to 639 of SEQ ID NO: 8.
[91] The method of any one of paragraphs [61]-[90], wherein the galactose oxidase is expressed from a heterologous polynucleotide.
[92] The method of any one of paragraphs [61]-[91], wherein the galactose oxidase is expressed from a host other than Fusarium austroamericanum.
[93] The method of paragraph [92], wherein the galactose oxidase is expressed from an Aspergillus oryzae host.
[94] The method of paragraph [92], wherein the galactose oxidase is expressed from a Fusarium venenatum host.
[95] The method of any one of paragraphs [61]-[94], wherein the galactose oxidase does not comprise the mature polypeptide sequence of SEQ ID NO: 2.
[96] The method of any one of paragraphs [61]-[95], wherein the reaction mixture further comprises a catalase.
[97] The method of any one of paragraphs [61]-[96], wherein the reaction mixture further comprises copper.
[98] The method of any one of paragraphs [61]-[96], wherein the reaction mixture further comprises copper sulfate.
[99] The method of paragraph [97] or [98], wherein the copper is at a concentration of less than or equal to 1 mM, e.g., less than or equal to 0.5 mM, or less than or equal to 0.0015 mM.
[100] The method of any one of paragraphs [61]-[99], wherein at least 10%, e.g., at least 20%, at least 30%, at least 40%, at least 50%, at least 60%, at least 70%, at least 80%, at least 90%, or at least 95% of the HMFCA or salt thereof is converted to FFCA or a salt thereof.
[101] The method of any one of paragraphs [61]-[100], wherein the reaction mixture further comprises a peroxygenase, and wherein the reaction mixture provides formylfuran carboxylic acid (FFCA), formylfuran carboxylic acid (FFCA), 2,5-furan dicarboxylic acid (FDCA), a salt thereof, or a mixture of the foregoing.
[102] The method of paragraph [101], wherein the peroxygenase has at least 60% sequence identity (e.g., at least 70%, at least 75%, at least 80%, at least 85%, at least 90%, at least 95%, at least 97%, at least 98%, at least 99%, or 100% sequence identity) to the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32.
[103] The method of paragraph [102], wherein the mature polypeptide sequence comprises the motif: E-H-D-[G,A]-S-[L,I]-S-R (SEQ ID NO: 27).
[104] The method of paragraph [101], wherein the peroxygenase has at least 60% sequence identity (e.g., at least 70%, at least 75%, at least 80%, at least 85%, at least 90%, at least 95%, at least 97%, at least 98%, at least 99%, or 100% sequence identity) to the mature polypeptide sequence of SEQ ID NO: 9.
[105] The method of paragraph [101], wherein the peroxygenase comprises or consists of the mature polypeptide sequence of SEQ ID NO: 9.
[106] The method of paragraph [105], wherein the mature polypeptide sequence is amino acids 1 to 328 of SEQ ID NO: 9.
[107] The method of any one of paragraphs [101]-[104], wherein the peroxygenase is a variant of a parent peroxygenase comprising a substitution at one or more (several) positions corresponding to positions 76, 134, and 201 of SEQ ID NO: 10.
[108] The method of any one of paragraphs [101]-[107], wherein the peroxygenase is a variant of a parent peroxygenase, comprising a substitution at a position corresponding to position 76 of SEQ ID NO: 10.
[109] The method of paragraph [108], wherein the substitution at a position corresponding to position 76 of SEQ ID NO: 10 is with Leu.
[110] The method of paragraph [108], wherein the substitution at a position corresponding to position 76 of SEQ ID NO: 10 is M76L.
[111] The method of any one of paragraphs [101]-[110], wherein the peroxygenase is a variant of a peroxygenase, comprising a substitution at a position corresponding to position 134 of SEQ ID NO: 10.
[112] The method of paragraph [111], wherein the substitution at a position corresponding to position 134 of SEQ ID NO: 10 is with Leu.
[113] The method of paragraph [111], wherein the substitution at a position corresponding to position 134 of SEQ ID NO: 10 is M134L or M127L.
[114] The method of any one of paragraphs [101]-[113], wherein the peroxygenase is a variant of a parent peroxygenase, comprising a substitution at a position corresponding to position 201 of SEQ ID NO: 10.
[115] The method of paragraph [114], wherein the substitution at a position corresponding to position 201 of SEQ ID NO: 10 is with Phe.
[116] The method of paragraph [114], wherein the substitution at a position corresponding to position 201 of SEQ ID NO: 10 is Y201F or Y194F.
[117] The method of any one of paragraphs [101]-[116], wherein the peroxygenase is a variant of a parent peroxygenase, comprising a substitution at any two positions corresponding to positions 76, 134, and 201 of SEQ ID NO: 10.
[118] The method of any one of paragraphs [101]-[117], wherein the peroxygenase is a variant of a parent peroxygenase, comprising a substitution at each position corresponding to positions 76, 134, and 201 of SEQ ID NO: 10.
[119] The method of any one of paragraphs [101]-[118], wherein the reaction mixture further comprises supplemental H₂O₂.
[120] A method of oxidizing 5-hydroxymethylfurfural (HMF), comprising contacting HMF with a peroxygenase in a reaction mixture under suitable conditions to provide 2,5-diformylfuran (DFF), 5-hydroxymethyl-2-furancarboxylic acid (HMFCA), formylfuran carboxylic acid (FFCA), 2,5-furan dicarboxylic acid (FDCA), a salt thereof, or a mixture of the foregoing.
[121] A method of oxidizing 2,5-diformylfuran (DFF), comprising contacting DFF with a peroxygenase in a reaction mixture under suitable conditions to provide formylfuran carboxylic acid (FFCA), 2,5-furan dicarboxylic acid (FDCA), a salt thereof, or a mixture of the foregoing.
[122] A method of oxidizing 5-hydroxymethyl-2-furancarboxylic acid (HMFCA) or a salt thereof, comprising contacting HMFCA or a salt thereof with a peroxygenase in a reaction mixture under suitable conditions to provide formylfuran carboxylic acid (FFCA), 2,5-furan dicarboxylic acid (FDCA), a salt thereof, or a mixture of the foregoing.
[123] A method of oxidizing formylfuran carboxylic acid (FFCA) or a salt thereof, comprising contacting FFCA or a salt thereof with a peroxygenase in a reaction mixture under suitable conditions to provide 2,5-furan dicarboxylic acid (FDCA) or a salt thereof.
[124] The method of any one of paragraphs [120]-[123], wherein the peroxygenase has at least 60% sequence identity (e.g., at least 70%, at least 75%, at least 80%, at least 85%, at least 90%, at least 95%, at least 97%, at least 98%, at least 99%, or 100% sequence identity) to the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32.
[125] The method of paragraph [124], wherein the mature polypeptide sequence comprises the motif: E-H-D-[G,A]-S-[L,I]-S-R (SEQ ID NO: 27).
[126] The method of any one of paragraphs [120]-[123], wherein the peroxygenase has at least 60% sequence identity (e.g., at least 70%, at least 75%, at least 80%, at least 85%, at least 90%, at least 95%, at least 97%, at least 98%, at least 99%, or 100% sequence identity) to the mature polypeptide sequence of SEQ ID NO: 9.
[127] The method of any one of paragraphs [120]-[123], wherein the peroxygenase comprises or consists of the mature polypeptide sequence of SEQ ID NO: 9.
[128] The method of paragraph [127], wherein the mature polypeptide sequence is amino acids 1 to 328 of SEQ ID NO: 9.
[129] The method of any one of paragraphs [120]-[126], wherein the peroxygenase is a variant of a parent peroxygenase comprising a substitution at one or more (several) positions corresponding to positions 76, 134, and 201 of SEQ ID NO: 10.
[130] The method of any one of paragraphs [120]-[126], wherein the peroxygenase is a variant of a parent peroxygenase, comprising a substitution at a position corresponding to position 76 of SEQ ID NO: 10.
[131] The method of paragraph [130], wherein the substitution at a position corresponding to position 76 of SEQ ID NO: 10 is with Leu.
[132] The method of paragraph [130], wherein the substitution at a position corresponding to position 76 of SEQ ID NO: 10 is M76L.
[133] The method of any one of paragraphs [120]-[132], wherein the peroxygenase is a variant of a peroxygenase, comprising a substitution at a position corresponding to position 134 of SEQ ID NO: 10.
[134] The method of paragraph [133], wherein the substitution at a position corresponding to position 134 of SEQ ID NO: 10 is with Leu.
[135] The method of paragraph [133], wherein the substitution at a position corresponding to position 134 of SEQ ID NO: 10 is M134L or M127L.
[136] The method of any one of paragraphs [120]-[135], wherein the peroxygenase is a variant of a parent peroxygenase, comprising a substitution at a position corresponding to position 201 of SEQ ID NO: 10.
[137] The method of paragraph [136], wherein the substitution at a position corresponding to position 201 of SEQ ID NO: 10 is with Phe.
[138] The method of paragraph [136], wherein the substitution at a position corresponding to position 201 of SEQ ID NO: 10 is Y201F or Y194F.
[139] The method of any one of paragraphs [120]-[138], wherein the peroxygenase is a variant of a parent peroxygenase, comprising a substitution at any two positions corresponding to positions 76, 134, and 201 of SEQ ID NO: 10.
[140] The method of any one of paragraphs [120]-[139], wherein the peroxygenase is a variant of a parent peroxygenase, comprising a substitution at each position corresponding to positions 76, 134, and 201 of SEQ ID NO: 10.
[141] The method of any one of paragraphs [120]-[140], wherein the reaction mixture further comprises supplemental H₂O₂.

1. A method of oxidizing 5-hydroxymethylfurfural (HMF), comprising contacting HMF with a recombinantly expressed galactose oxidase in a reaction mixture under suitable conditions to provide 2,5-diformylfuran (DFF).

2. The method of claim 1, wherein the recombinantly expressed galactose oxidase has at least 60% sequence identity to the mature polypeptide sequence of SEQ ID NO: 2.

3. The method of claim 1, wherein the recombinantly expressed galactose oxidase comprises or consists of amino acids 1 to 639 of SEQ ID NO: 2.

4. The method of claim 1, wherein the recombinantly expressed galactose oxidase is a variant of a parent galactose oxidase comprising a substitution at one or more (several) positions corresponding to positions 326, 329, 330, and 406 of SEQ ID NO: 2.

5. The method of claim 4, wherein the recombinantly expressed galactose oxidase variant comprises or consists of amino acids 1 to 639 of SEQ ID NO: 6.

6. The method of claim 4, wherein the recombinantly expressed galactose oxidase variant comprises or consists of amino acids 1 to 639 of SEQ ID NO: 8.

7. The method of claim 1, wherein the recombinantly expressed galactose oxidase is expressed from a heterologous polynucleotide.

8. The method of claim 1, wherein the reaction mixture further comprises a catalase.

9. The method of claim 1, wherein the reaction mixture further comprises copper.

10. The method of claim 1, wherein at least 10%, e.g., at least 20%, at least 30%, at least 40%, at least 50%, at least 60%, at least 70%, at least 80%, at least 90%, or at least 95% of the HMF is converted to DFF.

11. The method of claim 1, wherein the reaction mixture further comprises a peroxygenase, and DFF is further oxidized to formylfuran carboxylic acid (FFCA), 2,5-furan dicarboxylic acid (FDCA), a salt thereof, or a mixture of the foregoing.

12. The method of claim 11, wherein the peroxygenase has at least 60% sequence identity to the mature polypeptide sequence of any one of SEQ ID NOs: 9, 10, 11, 12, 13, 14, 15, 16, 17, 18, 19, 20, 21, 22, 23, 24, 25, 26, 28, 29, 30, 31, or 32.

13. The method of claim 12, wherein the mature polypeptide sequence comprises the motif: E-H-D-[G,A]-S-[L,I]-S-R (SEQ ID NO:27).

14. The method of claim 11, wherein the peroxygenase comprises or consists of amino acids 1 to 328 of SEQ ID NO: 9.

15. The method of claim 11, wherein the peroxygenase is a variant of a parent peroxygenase comprising a substitution at one or more (several) positions corresponding to positions 76, 134, and 201 of SEQ ID NO: 10.

16. A method of oxidizing 5-hydroxymethyl-2-furancarboxylic acid (HMFCA) or a salt thereof, comprising contacting HMFCA or a salt thereof with a galactose oxidase in a reaction mixture under suitable conditions to provide formylfuran carboxylic acid (FFCA) or a salt thereof.

17. A method of oxidizing 5-hydroxymethylfurfural (HMF), comprising contacting HMF with a peroxygenase in a reaction mixture under suitable conditions to provide 2,5-diformylfuran (DFF), 5-hydroxymethyl-2-furancarboxylic acid (HMFCA), formylfuran carboxylic acid (FFCA), 2,5-furan dicarboxylic acid (FDCA), a salt thereof, or a mixture of the foregoing.

18-20. (canceled)
